(19) United States
(12) Patent Application Publication
(10) Pub. No.: US 2017/0215807 A1
(43) Pub. Date: Aug. 3, 2017

(54) A CARDIAC STATE MONITORING SYSTEM

(71) Applicant: INOVACOR AB, Vaxholm (SE)

(72) Inventors: Stig Lundback, Vaxholm (SE); Jonas Johnson, Norrtaelje (SE); Fredrik Bergholm, Nynaeshamn (SE)

(21) Appl. No.: 15/501,262

(22) PCT Filed: Jul. 3, 2015
(86) PCT No.: PCT/SE2015/050784

§ 371 Date: Feb. 2, 2017

(30) Foreign Application Priority Data
Aug. 5, 2014 (SE) 1450923-6

(51) Int. Cl. A61B 5/00; A61B 5/11; G06F 17/30; A61B 8/08; A61B 5/02; A61B 5/026; A61B 5/021; A61B 6/00

(52) U.S. Cl. CPC A61B 5/7264; A61B 5/021; A61B 5/1107; A61B 6/503; A61B 8/0883; A61B 5/02028; A61B 6/5217; A61B 8/5223; A61B 5/7282; A61B 5/0022; A61B 5/0263; G06F 17/30867; A61B 2562/0219; G06F 19/321

(57) ABSTRACT

A cardiac state system, comprising a processing unit (4) configured to receive input signals (6) including parameters from, or related to, one or many registration points or areas within or outside a heart (8), and a storage unit (10) where one or many search tools are stored. The processing unit (4) is configured to process the input signals (6), by applying said search tools, to identify point of interests (POI), being landmarks, patterns and/or group patterns. The processing unit (4) is further configured to search for and identify global and/or regional event markers among said POIs to evaluate hydro-mechanical and/or hydro-dynamic functions of the heart. Preferably, at least some of said identified event markers are associated to the AV-piston defined according to the dynamic adaptive piston pump (DAPP) technology.

### FIELD OF THE INVENTION

The present invention relates to a cardiac state system, and a method in a cardiac state system, according to the preambles of the independent claims, adapted in particular for quantifying hydro mechanical and/or hydro dynamical cardiac timings and/or patterns.

### BACKGROUND OF THE INVENTION

Different medical equipment exists for monitoring the activity of the heart and circulatory system which may be based on e.g. large and advanced systems such as Magnetic Resonance Imaging (MM), ultrasound or electrocardiography.

Lately, also simpler and less expensive diagnostic devices have been introduced, applicable to be used by a physician or by the monitored person himself. The monitored persons may be patients or also persons performing various exercise activities, e.g. cycling, skiing, running.

Health care in the society faces large challenges by increasing costs and a growing elderly population that also is vital and well-read which set high demands on both the quality of delivered health care as well as the accessibility of health care related information e.g. regarding diagnosis and disease progression.

To meet the increasing demands on health care without drastically growing expenses, diagnosis, therapy and monitoring has lately been increasingly focused around healthcare carried out at home or in primary care, with focus also on pre-emptive measures such as wellness and lifestyle interventions. In these cases, when healthcare must increasingly be carried out in the peripheral parts of the healthcare system and with less specialist resources, it is desirable that there are tests and investigations to detect and monitor disease at e.g. a patients home and that these tests may easily and efficiently be correlated and compared to previous investigations performed at specialist centers such that the individual, a physician or personal trainer may easily evaluate and follow up results of e.g. different therapeutic interventions.

As examples of simpler investigation methods used today for e.g. “personal healthcare” are registration of blood pressure, heart sounds, respiratory rate, pulse oximetry and simple ECG-units.

None of these investigation methods are capable of obtaining a complete picture of the present state of the heart and circulatory system to e.g. diagnose or monitor myocardial ischemia or heart failure, and may not be fully correlated to more advanced investigations and diagnostic methods applied within the specialized healthcare.

Constantly developing sensor technologies have resulted in that there is now sophisticated monitoring equipment that is considerably smaller and more power efficient than earlier. One example is a pulsed Ultra Wideband radar. By providing such a radar chipset with small antennas adapted for detection of heart movements, it is possible to obtain measurements related to the heart's movements or movements of other internal organs or blood flow to evaluate physiologic parameters.

Such monitoring systems, based on e.g. radar or small handheld ultrasound scanners, could with proper processing and analysis of obtained signals prove to be valuable tools in assessing cardiac and circulatory functionality in new contexts. They could be both easy to handle as well as objective, applicable to be used in a wide range of the healthcare organization not only by specialist practitioners, but also for e.g. fast and easy screening of cardiovascular symptoms in emergency departments, primary care clinics or even by patients themselves.

The present invention is based upon and applies the discovery of previously unknown aspects of the pumping physiology of the heart presented in the thesis “Cardiac Pumping and Function of the Ventricular Septum” (Lundbäck 1986). This discovery showed that the heart, contrary to the dominating belief, does not work as a squeezing displacement pump but rather according to a new class of pumps with different properties than any earlier known pump type. This has emerged into a new pumping technology which is today called the Dynamic Adaptive Piston Pump (DAPP) technology characterized by a unique piston construction operating as the main pumping unit where said piston has central and peripheral differential areas (deltaV-areas) between the inflow and outflow chambers giving the pump unique properties which cannot be achieved with prior art pumping technology.

A general implementation of this technology is covered by U.S. Pat. No. 7,239,987. In patents EP-1841354, U.S. Pat. No. 8,244,510 and in the patent applications EP-2217137 and WO-2007/142594, the DAPP-technology has been used to model the pumping and hemodynamic controlling functions of the heart by representing the heart as a “Cluster State Machine” comprised by many small unit machines, the heart muscle cells, that together create a compound pump working in accordance with the pump mechanical principles of the DAPP-technology. The mechanical features of the compound pump are described as a state diagram.

Thus, based upon the knowledge of the true mechanics of the heart, e.g. its pumping and controlling functions, it is possible to establish a so-called “Cardiac State Diagram” (CSD). This diagram makes it possible to illustrate mechanisms that, in a time-related order, control the mechanics of the heart. The DAPP-technology may via a CSD, in a correct time order, and without a specific starting point, decode and visualize values that both advanced and simple investigation methods may generate.

In addition, a CSD may be applied to establish a bridge or connection between advanced investigation methods and simpler methods.

The sensor technology has been developed and monitoring equipment that used to be very energy consuming and large is now considerably smaller and less energy consuming. One example is a radar sensor chip. By providing the chip with small antennas adapted for detection of heart movements it is possible to obtain measurement values related to the heart's activity and also breathing frequency, etc.

In the patents and patent applications referred to above it has been described that CSDs may be established from information in signals obtained both from internal sensors (within the body) and external sensors. Since internal sensors have better possibilities to register absolute values related to cardiac mechanical activities such as e.g. pressure, these signals may be used to relate and validate parameters in a CSD quantified from external sensors.

The algorithms used for the decoding procedure should of course result in essentially the same Cardiac State Diagram irrespectively if it was established with advanced investigation methods such as ultrasound equipment, MR, CT, or if it was established by simpler methods, such as small radar sensors, accelerometers, pressure sensors, etc.

In the patents/patent applications referred to above it has been described that, based upon the DAPP-technology, it is possible to establish Cardiac State Diagrams (CSD). A CSD, in combination with reference databases, can form basis for decisions regarding diagnosis, treatment and follow-up both for specialist users (e.g. physicians) and for less experienced users within heart healthcare.

In this regard it is important to declare that the total heart is servicing two circulatory systems, except the heart's own circulatory system that are in a serial connection to each other. This means that the heart's global functions described by the CSD must reflect how the interactions between these two circulatory systems, though high differences in pressure, can maintain a circulatory balance and maintain low filling pressures though handling a wide range of flows and frequencies.

There are many disturbing factors such as corrupt signals and misleading motion artifacts that both jeopardize the findings of global and local hydro mechanical and/or hydro dynamical timings in signals associated to the hearts activities.

A very common misleading movement artifact is the heart's fully normal motion pattern in at least five axes of motion. These movement artifacts are results of that the point or points used for detection during the detection procedure are not the same point(s) throughout the whole procedure and that they in addition most surely are seen from a different angle of view.

The motion/velocity changes that on the first glance can be assumed to be artifacts can also be true changes. That depends on that the heart muscle cells are elastically tied together resulting in that malfunctions of heart muscle cells at the investigated point/area can, through their elastic links overrule, mask, delay or bring forward event markers that might not represent the sought after events used to set up CSD at the investigated point/area.

When implementing the DAPP-technology for establishing a Cardiac State Diagram it is sometimes, e.g. for the above mentioned reasons, difficult and time-consuming both to identify and to determine the exact timing of event markers in order to establish a CSD and further to decide what kind of impact regional activities in various registration points/areas exert on to the CSD.

The reason is that the enormous amount of data that should be transformed, decoded, e.g. as suggested above, is subjected to distortions, and wrong projections, that often changes vastly during e.g. increase or decrease of frequency for input and/or output flows to the heart, and also during all kinds of heart failure.

To build and establish reliable reference databases only by using detailed time markers is difficult. The reason is that the CSD, that is supposed to decode the global function of the heart, not always is established with the correct time markers to accurately represent a global function; it might just be a representation of local activities and or disturbances.

The above discussions result in a conclusion that there is still room for further improvements of the procedure used to find local and global time markers and patterns used to classify local and global hydro mechanical or hydro dynamical activities in order to establish a Cardiac State Diagram and to assess its mechanical background.

Thus, the object of the present invention is to achieve a system for improved processing of signals related to the heart's activities in order to find local and global time markers and patterns to establish a classification of local and global hydro mechanical and hydro dynamical activities. Thereby it is possible to further increase the applicability and use of CSD and assess its underlying local activities for improving e.g. diagnostic and therapeutic methods.

### SUMMARY OF THE INVENTION

The above object is achieved by the present invention according to the independent claims.

Preferred embodiments are set forth by the dependent claims.

The present invention relates to a cardiac state system, configured to decode, determine, and classify the mechanical functions of a heart during one or many heart cycles, based upon sensed parameters from, or related to, one or many registration points or areas within or outside the heart to identify local/sub activities at different points/areas, regions inside/outside the heart and its vessels and to classify these activities participation in the global heart mechanical functions in accordance to a the rule based model which is an event and timing function rule based model. Preferably, the rule based model is according to the DAPP-technology.

This improves the possibilities to verify, differentiate and classify the CSD and also to verify what kind of deviation activities in different regions have from real and/or expected global functions.

More specifically, the system is configured to receive and process input signals obtained from advanced investigation methods where the heart functions can be derived from complex series of images, e.g. ultrasound, computed tomography, or MRI, and/or from less advanced investigation methods, e.g. pressure- and flow-sensors, accelerometers, or radar sensors.

In particular the present invention includes embodiments that relate to a system configured to detect and evaluate local mechanical performance represented as Local Function Parameters (LFP) and/or global mechanical performance represented as Dynamic Factors (DF), e.g. by using pattern recognition and/or a timing framework. Further embodiments are included configured to determine State Indexes (SI) for further highlighting deviations in cardiac mechanical performance.

The invention relates to solving three distinct problems.

The first problem is to correctly define the time markers and patterns used to establish a CSD and review its underlying mechanical activities. This problem consists both of formulating the purely physiological definitions for the sought after cardiac mechanical events as well as defining how these pump physiological or mechanical events are reflected in captured signals. A significant aspect of this problem has shown to be that it is difficult to determine if an identified event marker or pattern is of global or local character.

The second problem consists of how to achieve a robust procedure for identifying the defined event markers and patterns in the captured signals by use of e.g. search algorithms.

The third problem concerns the analysis and interpretation of the parameters obtained. For example how to combine and/or relate timing parameters and/or non-timing parameters to achieve State Indexes (SI) for further highlighting deviations in cardiac mechanical performance in order to e.g. maximize sensitivity to detect cardiac pathologies.

### SHORT DESCRIPTION OF THE APPENDED DRAWINGS

FIGS. 1a-d show long axis views of the heart, illustrating AV-piston motions, equator line, DeltaV-area and RsA changes and DeltaV- and RsA-volume changes, respectively.

FIG. 2a-d show short axes views of the heart, illustrating AV-piston motions, DeltaV-area and RsA changes, and DeltaV- and RsA-volume changes, respectively.

FIG. 3 is a schematic illustration of a cardiac state system according to the present invention.

FIG. 4 is a flow diagram illustrating a method in the cardiac state system according to the present invention.

FIG. 5 is a detailed flow diagram illustrating different processing steps according to embodiments of the present invention.

FIG. 6 illustrates various examples of detection methods (denoted A-D) for detecting different movement patterns of the heart.

FIG. 7 is a schematic high level illustration of the GrippingHeart Platform (GHP).

In FIG. 8 is shown examples in the form of input signals of measured velocity and accelerations during one heart cycle.

FIG. 9 illustrates one curve segment where POIs have been indicated.

FIG. 10 illustrates examples of calculating state indexes.

FIG. 11 is a flow diagram illustrating the method of establishing a CSD.

FIG. 12 shows a schematic illustration of one embodiment where the measurements are made by a small radar sensor unit.

FIG. 13 is an illustration of how the Cardiac State System may interact with other systems in the GrippingHeart Platform (GHP) to support simulations.

### DETAILED DESCRIPTION OF PREFERRED EMBODIMENTS OF THE INVENTION

The present invention will now be described in detail with references to the appended figures.

Throughout the figures the same or similar items and/or functions will have the same reference signs.

In order to fully cover all aspects of the present invention and with the intention to make it best understood the description is divided into two parts.

The first part is related to a rule based model of how to analyze and classify the heart mechanics in accordance with the DAPP-technology, to be used as guidelines for achieving a structured timing and pattern recognition framework described in part 2 that can be used to find event markers and patterns for local and/or global hydro mechanical/dynamical functions of the heart, e.g. for establishing CSDs.

Thus, the second part is a pattern and timing recognition system structured to analyze and classify the heart's mechanics according to a model of the heart having an AV-piston construction in accordance to the DAPP-technology.

The structured model of part 1 for analyzing and classifying heart mechanics in combination with the pattern recognition framework of part 2 is to be part of a platform, GrippingHeart Platform (GHP) that may perform one or many of:
Detect, classify and differentiate local hydro mechanical timing and patterns from global timing and patterns out of heart related signals.
Connect to reference databases to store and make use of generated data and information to further support timing and/or pattern recognition out of heart-related signals.
Provide decision support in the evaluation of local and/or global hydro mechanical/dynamical functions of the heart.
Provide support for treatment and monitoring of cardiac diseases.
Support simulation of pharmaceutical and surgical treatments and outcome thereof.
Support monitoring and optimization of overall circulatory performance for e.g. wellness activities.
Support and increase the applicability of other systems and methods for studying mechanics of the heart, e.g. external torsion of the heart, aorta and pulmonary artery or internal torsion in the heart muscle etc.
Support and increase the applicability of other systems and methods that are associated to studying the central and/or peripheral circulatory system e.g. pulls and propagation intensity/velocities in vessels such that these can be compared and related to the mechanics of the heart.
Support and increase the applicability of other systems and methods used to model and simulate the heart and circulatory system.

Part One: Background

The CSD was intended to describe the overall, global mechanical function/hydrodynamics of the heart, but it has proven to be rather difficult to accurately find time markers of global heart events to set up a CSD when using current guidelines for routine investigations of the heart. Current investigation procedures most often just concern the function of the left ventricle and local muscular activities. This means that the muscular activities used to assess cardiac functionality are not directly linked to the heart's global mechanical performance.

It has been proven that establishing a CSD by calculating the mean value from different local points and/or areas of the heart to assess the heart's global functions is possible when the heart is working under normal physiological conditions. But as soon as the heart is not working normally resulting in disturbed hydromechanics, as when the heart is subjected to cardiac disease, mean values will no longer accurately represent global function.

Under these circumstances, it has been shown that comparing differences in timing of local hydro-mechanical events during the cardiac cycle pre-ejection phase (as defined according to the DAPP-technology) is more sensitive to identify heart disorders than global CSD time intervals calculated by mean values of the same local events.

One possible hydro-mechanical explanation to these findings is that since the pressure during the pre-ejection phase is not high enough to stabilize the shape of the ventricular and AV-plane geometry, deviations in hydro-mechanical performance between investigated sectors will not be carried onto opposing and neighboring sectors and thus makes time markers and patterns seen during this phase very robust for assessing local functions (see further below). This is an example of how a model with rules and definitions of cardiac mechanical events can be used to find, validate and classify local and global event markers in signals.

In a CSD established by calculated mean values, local deviations will per definition to a certain extent be neutralized and the sensitivity to assess local hydro mechanical function may thus be low.

Part One: Summary

Part one describes a model of how the different tissue and/or hydro mechanical forces in the heart and circulatory system interact as well as how this interaction changes over time throughout the mechanical chain of events in the cardiac cycle. To achieve this, the heart is modelled as a piston pump operating in accordance to the DAPP-technology, to give guidelines for detection, validation and classification of local and global functions of the heart.

With the DAPP-technology as a model of the heart's mechanics it is possible to e.g. better find and to classify relations between motions and counteracting forces inside and/or outside the heart to accurately identify and differentiate local and global event markers and patterns.

This will increase opportunities to find event markers that are depicting local mechanical performances of the heart during time intervals where there is no high pressure stabilizing the cardiac geometry (which would spread forces evenly in the heart). It will also during time intervals with high pressure such as during ventricular ejection facilitate finding of patterns reflecting the summarized global performance of the heart.

Motions of the AV-piston, IVS and other structures inside and outside of the heart including the great vessels as well as the blood flow into, through and out from the heart are all depending on the forces generated by the contraction elements in the heart muscle cells. These primary forces which are transmitted through elastic links in the muscle tissue will also create secondary counteracting forces according to Newton's third law of motions. These forces will contribute to a pattern of balance that can be used to find and validate event markers in signals with possibilities to find and differentiate local hydro mechanical functions from global hydro mechanical/dynamical functions.

By using part one to form rules, conditions and definitions for the search tools described in part two, it will be possible to robustly and accurately search for event markers and patterns in points/areas that e.g. by the construction and motions of the AV-piston generate counteracting balancing forces.

These conditions can be established for signals acquired both from points inside and outside the heart.

Inside the heart, these conditions can e.g. be related to the balancing motions of the AV-piston that by e.g. sliding and rotation inside the pericardial sack adjusts its motions to the forces that are exerted on it.

Outside the heart, these balancing functions can e.g. be found as volume changes arising above and below the heart with resulting external tension forces that will create event markers and patterns that are more orientated to the global functions of the heart.

Further time events that are associated with the global function of the heart are those that are linked to the motions of the IVS (Intra Ventricular Septum) and also of course the blood flow entering or leaving the heart.

Part one could also provide a basis for how to optimize placement of registrations points (regions of interest, ROI) to acquire rich signals containing event markers and movement patterns reflecting both the heart's local and global functions.

With this model, with or without reference databases, it will be easier to understand and classify how influences of local activities, e.g. collected from the left ventricle, will affect the heart's global function, the CSD, or vice versa.

Part Two: Summary

Part two is an example of a timing and pattern recognition framework that can be used to find event markers and patterns for local and/or global hydro mechanical/dynamical functions of the heart represented as a CSD.

Part two, based on guidelines from part one describes a concept how to detect, validate and analyze the heart's local and global hydro mechanical and hydro dynamical functions. In addition the concept supports classification of local heart functions with Local Function Parameters and/or global heart functions with Dynamic Factors and also supports the determining of State Indexes (SI) for further highlighting deviations in cardiac mechanical performance.

Part two and part one, are integrated in a platform that is configured to acquire, differentiate, organize and classify local and global event markers and patterns in reference databases that iteratively can be used to decode heart related signals and as a decision support tool for heart related diagnosis and therapy.

In order to support the above mentioned wide range of possibilities to detect and classify local and global functions of the heart by e.g. balancing counteracting forces, the heart's hydro-mechanical/dynamical functions modeled according to the DAPP-technology has to be explained in more details.

Part One: A Detailed Description

Part one is a model of the heart as having a piston construction working in accordance to the DAPP-technology to give guidelines for establishing rules, conditions and definitions to detect, validate and classify local and global functions of the heart.

The heart described as a pump according to the DAPP-technology.

A detailed description of the heart as a pump according to the DAPP-technology will now follow, that may also be regarded as detailed guidelines applicable when implementing the present invention.

The heart's structure and function can be modelled as a piston pump according to the DAPP-technology which among other things explain how inflow of blood to the heart under low, more or less constant static filling pressures is distributed into the heart under its systolic and diastolic phases and creates the heart's inflow controlled auto-regulating properties.

The below points define from a mechanical point of view which conditions regarding the heart's anatomy and function that must be fulfilled in order for it to be described according to the DAPP-technology:
There must be an AV-piston with deltaV-areas that generate external deltaV-volumes.
The AV-piston must be able to slide freely inside the pericardium like a “cylinder function”.
The heart musculature constitutes both the structure and the source of power in the heart as a pump.
The heart musculature can by developing force in one direction transmit energy both to the heart's inflow and outflow.
The geometry of the AV-piston allows it to apart from creating flow through the heart, also transfer energy to its surroundings which among other things is necessary to create an uninterrupted inflow during the period where the piston starts to change direction and during its return.
The AV-piston will by the aid of the stored energy have a hydraulically controlled return which becomes adapted to the inflow.
The muscle cells way of in a contractile state stabilize (systole) and in a relaxed state destabilize (diastole) its muscular structure means that the IVS, being a partition wall between the right and left ventricle, in principle becomes dissolved and thereby the AV-piston can be regarded as one common piston for both the right and left side of the heart with possibilities to balance the filling of the heart from both the systemic and the pulmonary circulation.

The above points will now be explained in more detail.

Anatomical conditions that must be fulfilled for the heart to be able to operate as a pump constructed in accordance with the DAPP-technology

The Pericardium

The pericardium is a somewhat foldable (flexible/deformable) but not very stretchable fibrous sack which demarcates the myocardium's outermost layer, the epicardium, toward the surrounding tissues. As a result of its properties, the pericardium will under normal static filling pressures delimit a more or less pre-determined maximum volume for the heart's tissues and its content of blood.

Its egg-like shape and alterations of this shape is to a large extent determined by three points of fixation to the surroundings, the construction of the AV-piston with atrial and ventricular volumes as well as the central stabilizing unit being the Intraventricular Septum (IVS). Through movement of these structures and the pericardium, volume changes will be created above and below the heart which in turn results in tension forces and external compliance volumes that will enclose the pericardial sack.

Between the epicardium and the parietal layer of the serous pericardium there is a thin layer of fluid which facilitates the movement of these two layers against each other so that the heart under normal conditions can slide inside the pericardial sack with very low friction.

The Three Fixations Points of the Pericardium

The surroundings of the pericardium can be said to form three areas of fixation which orients the pericardium's possibilities for global motions.

Fixation Area 1:

The pericardium has an upper, basal calotte-shaped form which is moderately attached to the surrounding through the inflow vessels vena cava inferior and superior entering the right atrium and the pulmonary veins entering the left atrium. The pericardium's basal form furthermore borders the pulmonary arteries which lie in close connection to the spinal column. The pericardium and thereby also the basal plane of the heart thereby forms a moderately firm attachment to its surrounding. The aorta leaves the pericardial sack in the form of a rolling diaphragm-like function generated by the pericardium.

Fixation Areas 2 and 3:

The pericardial sack does after its calotte-shaped base extend to form an egg-shaped volume which encloses the entire heart. This egg-shape, which among other things is determined by the activities of the AV-piston, has a surface adjacent to the diaphragm through which the pericardium is firmly attached to the diaphragm aponeurosis. This connection is in some anatomical literature referred to as the “phrenopericardial ligament”. The pericardium further has a surface following the thoracic wall by which it is more or less hydraulically locked to, making the pericardium and thoracic wall inseparable under normal circumstances. This confers that the pericardial sack can only move parallel to the thoracic wall with conjoint movement of the diaphragm. This works well under expiration and inspiration when the pericardium and the heart with ease can follow the up and down respiratory motions of the diaphragm. Sideways however, the pericardium's attachment to the diaphragm will strongly limit the heart's possibilities to move sideways along the thoracic wall.

In all other areas the pericardial sack is more or less surrounded by lung tissue.

The three fixation points and support from the pericardial sack and its surrounding structures described above is of crucial importance for the back and forth motions of the AV-piston and its impacts on the heart's surroundings.

The Heart's Hydraulic Attachment to the Pericardium

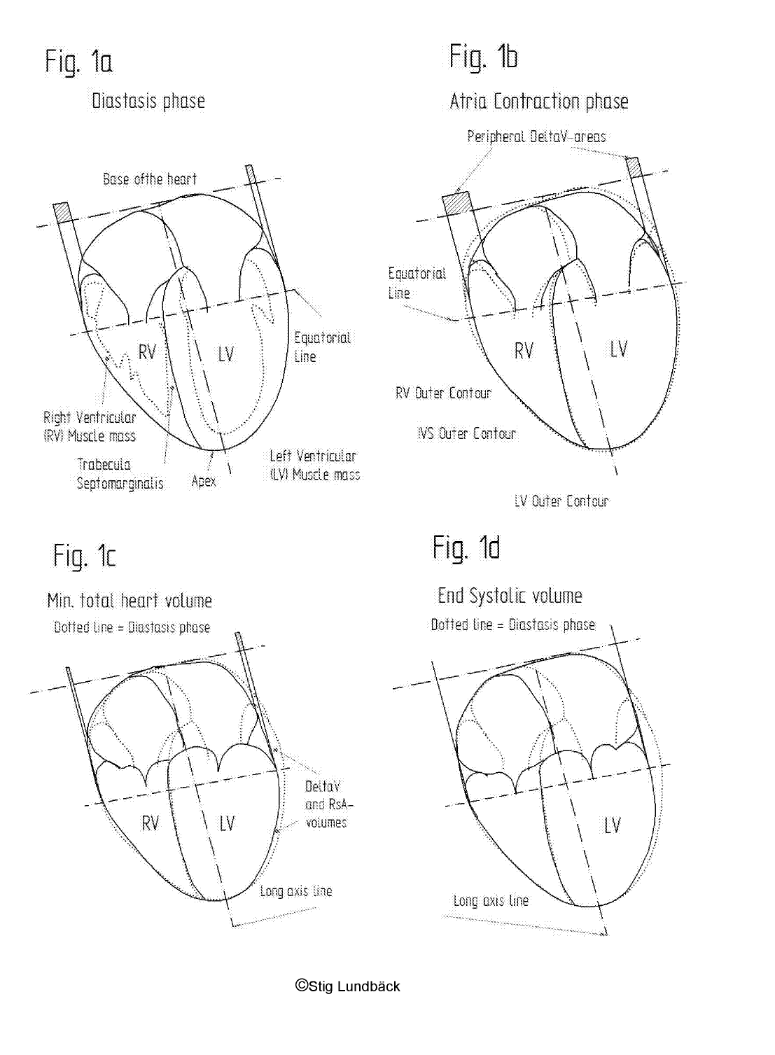

Both the ventricular and the atrial musculature have volumes that are more or less the same whether the muscle is in a contracted or a stretched out state. Therefore all the heart's volumes made up of muscle tissue can be seen as outer contours enclosing both muscle and blood volumes (FIG. 1b, 2b-d) The structure of the AV-piston and its division of the heart into atrial volumes and ventricular volumes as well as its motions and its effects on the egg-shape of the pericardium will by this illustration become easier to understand.

The upper, calotte-shaped basal form of the atrial musculature is, like the pericardium in the same region, firmly attached to the inflow vessels vena cava inferior and superior whose attachments to the heart constitutes a part of the right atrium's upper delimited surfaces and volumes.

Truncus Pulmonalis is situated inside the pericardial sack and does together with the outgoing of the Aorta from the AV-piston, which is also situated inside the pericardium, form a large part of the division between the right and left atrium.

The both outgoing vessels are surrounded by folds and flaps belonging to both the right and left atrium.

Following the pericardium's further extension over the atrial volumes, the entering of the four pulmonary veins in to the left atrium will further fixate the heart and the pericardium's basal calotte-shaped from to the surroundings.

Except from those areas where the inflow vessels enter the heart, the atrial volumes are hydraulically fixed to the pericardium.

The heart's base plane, in conjunction with the pericardium, therefore constitutes a firm attachment to a not very resilient surrounding.

The pericardial sack does after its calotte-shaped base extend to form an egg-shaped volume which encloses the entire heart, see further below. The heart is hydraulically attached to the pericardium, meaning that all heart volumes, except from those that are connected to the inflow vessels in the base of the heart, can slide and rotate along the shape of the pericardium, but not be separated from it.

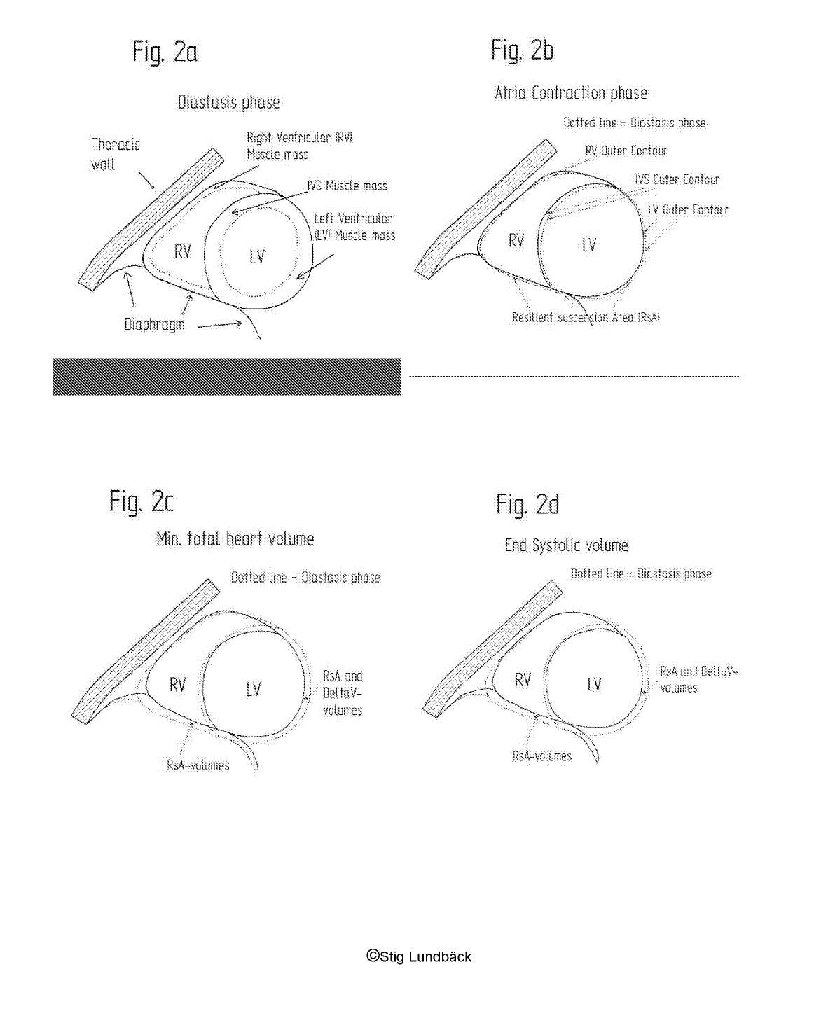

The heart's hydraulic attachment to the pericardium and its fixation points toward the thoracic wall means that the heart just like the pericardium has very little possibilities to globally move sideways. There are however possibilities for all kinds of sliding and rotational motions inside the pericardium. Furthermore, there are under certain conditions possibilities for the heart, through form- and volume changes of the pericardium, to move despite the pincher-like limitation that the thoracic wall and the diaphragm has on particularly the right ventricle's lower, cone-shaped part (FIG. 2a-d). This means that particularly the left ventricle including the IVS has some possibilities to expand and or move in and out of this pincher-limitation

The Pericardium and the Heart's Equatorial Line

To further enhance the understanding of the AV-piston's structure, forms, motions and their effects on the heart's surroundings over time, the egg-shape of the pericardial sack is divided by en equatorial line through its largest waist diameter, which thereby divides the heart in one upper and one lower egg-half volume (FIG. 1a-d).

By introducing an equatorial line which dynamically changes its circumference, which may be difficult to delimit in practice, there is however a practical and illustrative basis created for further theoretical descriptions of the heart's functions according to the DAPP-technology.

Dividing the heart by an equatorial line creates an ending on the AV-pistons, rounded muscular connection to the ventricles lower cone-shaped volumes.

Furthermore this division can define upper and lower egg-shaped volumes that by change in form and or position can create upper and lower external volume changes connected to the pericardium that generate tension forces (FIG. 1b-d, 2b-d). These will to a high extent form basis for the heart's functions and can be the subject of registration of event markers and motion patterns which will be further discussed below.

The force development in the cardiac musculature with longitudinal shortening of the outer contours.

The heart musculature is largely, from a mechanical perspective, built up of contractile and elastic components. When the ventricular musculature depolarizes, the contractile elements are subjected to calcium ions which trigger a shortening of the contractile elements that via shortening and thickening exert forces to the elastic elements that among other things result in mechanical work.

Without delving deeper into how nature through spiral- and helix formations solves the logistic problem of arranging the muscle cells so that they under shortening and concurrent thickening in several cell layers can cooperate in an optimal way, the ventricular contractions in principle results in that the ventricular outer contours are shortened longitudinally which displaces and moves both ventricular musculature and blood in direction of the apex.

The heart musculature's force development does already during the first half of the systolic ventricular ejection phase reach its maximum (without counting the force developed during the pre-ejection phase), see further below, and does thereafter start to decline by decreasing intracellular calcium concentrations.

This results in that the ventricular musculature's energy development to drive the AV-piston and thereby also blood out of the ventricles decreases as to finally lead to a ceased pulling of the AV-piston toward the apex. When the AV-pistons motions toward the apex start to diminish and finally ceases the inflow to the heart will start to decrease as well which is of negative consequence for the overall dynamics of the heart's internal filling. This is especially pronounced during high flow and frequency. Nature has however solved this problem by the structure and form of the AV-piston as well as its adaption to the egg-shape of the pericardium.

The Structure of the AV-Piston and its Motions Give Rise to Upper and Lower Compliance Like Functions with Upper and Lower External Tension Forces

The Heart's Piston, the AV-Piston

In the medical literature there is often described, somewhat simplified, that the heart is divided into atrial and ventricular volumes by a plane, the AV-plane, which most often is defined as the area created by the fibrous skeleton and the mitral and tricuspid valves which are fastened into this ring structure. Motions of this plane are described are termed AV-plane motions.

In this description we will use a wider definition of the anatomy that divides the heart into atria and ventricles called the AV-piston. Since the heart is working according to a piston pump principle it is important to define all structures included in the heart's piston unit.

Anatomical Structure of the AV-Piston

The AV-piston forms a common piston for both the right and left side of the heart which divides the heart into atrial and ventricular volumes. It consists of a central, more or less flat surface, the fibrous skeleton, forming a valve plane, the AV-plane. It contains the heart's four valves, two inflow valves and two outflow valves that are enclosed by the two outgoing vessels Truncus pulmonalis from the right ventricle and the Aorta from the left ventricle. The outflow valves do with their enclosing vessels on each side of the ventricular septum (IVS) form the AV-pistons central DeltaV-areas which are constant throughout the cardiac cycle.

The AV-piston does also consist of peripheral rounded surfaces which connect to the pericardium and to the ventricular musculature's cone-shaped volumes. This connection is comprised of the heart's equatorial line. The rounded surfaces of the AV-piston that are not covered by volumes originating from the atria constitutes the AV-pistons peripheral deltaV-areas (FIG. 1a-d). The IVS divides the AV-piston into one right and one left ventricular part, but they will be regarded as a common piston since the interactive functions that the IVS transmits between the right and left ventricle more or less causes the ventricles to under their inflow-controlled, auto-regulating time intervals to behave as one large, single volume enclosed by the pericardial sack, see further below.

The AV-piston's central surface with central deltaV-areas does by motion create central deltaV-volumes and results in upper, external tension forces.

The AV-piston has a central, flat area that consists of the heart's fibrous skeleton that housing two inflow- and two outflow valves where the two outflow valve's total surfaces and their related outflow vessels, on each side of the ventricular septum (IVS), forms the AV-pistons central deltaV-areas. These does by the motions of the AV-piston generate central deltaV-volumes and by the attached vessels resistance to tension and motion a part of the upper external tension forces developed, see further below. The central deltaV-areas with their outgoing vessels do only to a small part contribute to the filling of the atria through their connection toward small atrial volumes interspersed in between them.

The form of the AV-piston's peripheral rounded surface constitutes a part of the ventricular volumes upper muscular limits and does together with the AV-valves and its supporting papillary muscles create the classic symbolic heart-shape.

The ventricular musculature connects to the fibrous skeleton that is the AV-piston's central flat surface, by rounded surfaces that also constitutes a part of the ventricular volumes upper volume enclosing. The AV-piston's peripheral ending is defined to be at the equatorial line of this rounded form that is formed by the ventricular volumes largest outer diameter. It is natural to think that the AV-piston's fibrous skeleton gives rise to cross-connections resulting in that the AV-piston's muscular extension gets a rounded shape when the ventricular volumes are subjected to pressure simultaneously as the musculature transitions to form the ventricles cone-shaped lower parts (compare with the shape of an expanded parachute).

For the AV-piston to further keep its rounded shape and width when the entire AV-piston is subjected to pressure, it receives support from the truss structure that is formed by the papillary muscles and their chordae tendinae attachments to the atrioventricular valves. These structures form, if the IVS and the atria are disregarded, a configuration which much resembles the symbolic illustration of a heart's shape.

The AV-piston structure and the musculatures logistical arrangement means that the peripheral muscular part of the AV-piston both forms as well as follows the largest diameter of its width, which is its equatorial line. During the AV-pistons motions toward the apex this equatorial line will also move towards apex simultaneously as its radial width decreases (FIG. 1a-d).

The pericardium's basal calotte-shaped fixation toward its surroundings, its hydraulic attachment to the thoracic wall and its elastic attachment to the diaphragm, see further below, does together with, the structure, form, motions and pressurization of the AV-piston, give the pericardial sack its egg-shaped form and volume adjustments.

The atrial volumes connection to the AV-piston.

The atrial musculature also springs from the fibrous skeleton. They have a peripheral extension that directly via the auricular appendages and indirectly via fatty tissue forms a wedge that covers a large portion of the AV-piston's peripheral rounded parts. This fatty tissue which has is a flexibly built up and forms an adaptable wedge structure containing vessels. These wedge-like structures are hydraulically attached to both the rounded surfaces of the AV-piston as well as the pericardium's upper egg-like form.

This wedge of fat is also hydraulically attached to the atrial musculature. This structure does in conjunction with the fixation of the heart's base plane, confer that the AV-pistons motions toward the apex creates forces that force the atrial volumes to expand in their periphery and pull the AV-plane toward the base plane during atrial contraction

The pericardial sack's three points of fixation toward its surrounding confers that the peripheral deltaV-areas of the AV-piston generates upper peripheral deltaV-volumes with resulting upper external tension forces

The part of the AV-piston which directly or indirectly via the fat wedge and solid muscle wedges from the atria, borders the pericardium and thereby is not covered by structures that not includes atria blood-volumes, forms the AV-pistons peripheral deltaV-areas (FIG. 1a-d) These areas does upon motion of the AV-piston toward the apex and by the fixation of the base plane to the surroundings create peripheral external deltaV-volumes resulting in upper external tension forces. These forces the wedge-shaped peripheral atrial volumes to expand which means that energy may be added to the inflow of the atria. The peripheral deltaV-areas will continuously during the AV-piston's motions toward the apex create peripheral deltaV-volumes with resulting external tension forces above the dynamically changing equatorial line.

The upper external deltaV-volumes will with their associated upper external tension forces contribute to uphold a continuous inflow to the atria during ventricular systole as well as the period where the AV-pistons changes direction. Together with the central deltaV-volumes and their associated external tensions forces the peripheral deltaV-volumes with their associated tension forces will add energy during the fast filling phase and to the inflow-adapted hydraulic return of the AV-piston as well as furthermore receive energy to uphold inflow during the slow filling phase. See further below.

Summary of the Upper External and Internal Tension Forces that are Created During the AV-Piston's Motion Towards the Apex

The upper external tension forces are created when the AV-piston is pulled towards apex and are comprised of:
Peripheral and central deltaV-volumes with resultant tension-forces.
Longitudinal pulling and stretching of the outgoing vessels.
Stretching of the atrial musculature.
Acceleration of the inflow into the atrial volumes.
Friction forces.

The pericardial sack's three points of fixation towards its surroundings confers that the AV-piston's upper tension forces created by the contraction forces of the ventricular musculature receives counteracting, balanced forces below the equatorial line which together are termed the heart's resilient suspension (Resilient suspension Area, RSA) with RsA-volumes and associated resultant lower external tension forces.

Since the AV-piston extends to form the ventricular volumes cone-shaped volumes and these, except from the ventricular septum (IVS), are hydraulically fixed to the pericardium, there must be lower external tension forces created below the equatorial plane so that the ventricular volumes cone-shaped volumes are not pulled toward the AV-piston uninhibited during the ventricular systolic phase.

The outgoing vessels that are fixated to the AV-piston have their outflow areas inside the ventricular volumes in close proximity to the IVS which separates the both ventricles. During the pulling of the AV-piston toward the apex the outgoing vessel's walls will develop resistance when they together with the AV-piston are pulled toward the apex. Their angle-(Truncus Pulmonalis) and screw-shapes (Aorta) can reduce the need for stretching the vessel walls by the heart doing a slight mechanical rotation inside the pericardial sack.

The resistance from the expansion and the filling of the atrial volumes is together with the tension in the outgoing vessels transmitted by the ventricular musculature including IVS and enforced by Trabecula Septomarginalis, toward the pericardial sack's two fixation points to the thoracic wall and the diaphragm.

These fixation points can under certain conditions create lower external volumes with resulting external tension forces beneath the equatorial line. These can match and balance the tension forces that are created by the AV-piston's motions above the equatorial line. The forces created beneath the equatorial line are therefore described as RsA (Resilient suspension Area) that by their RsA-volumes together with the AV-piston's deltaV-volumes and their associated upper tension forces gives a balanced inflow into the heart.

The outgoing vessels that are fixated to the AV-piston and their close connections to the IVS get a direct connection to the diaphragm. The angled exits of the vessels also affect the motions of the left ventricles posterior-lateral surface which may be one of the reasons that the RsA-volumes seem to be largest within this region.

The pericardial sack's attachment to the diaphragm is not as rigid as its basal fixation towards the surrounding, which means that the diaphragm can be pulled up towards the piston and reduce its stroke length in the apical direction, resulting in both positive and negative effects, see further below.

The upper and lower external volume changes with associated tension forces can also be described as upper and lower external compliance volumes.

The upper external deltaV-volumes with their resulting tensions forces arises as a consequence of the AV-pistons structure and motions and can, besides the compliance volumes in the inflow vessels, be said to form the heart's upper external compliance-like volumes. The lower external RsA-volumes and their resultant tension forces are created as an effect of the upper and can be said to form the heart's lower compliance-like volumes.

The interplay between the AV-pistons motions, the upper and lower external volume changes and their resulting tension forces creates balanced event markers and motion patterns.

Between the two upper and lower counteracting external tension forces described above is the cardiac musculature. This means that the cardiac musculature except from creating internal tension forces to generate flow and pressure also must contain tensions forces that may create a bridge between the upper and lower external tension forces.

The ventricular musculatures contractile elements will through the ventricular musculature's contractions and sliding motions along the pericardial sack connect different regions/segments of the heart with each other. Also, these regions will connect the upper and lower external volumes created and their resultant tension forces with each other.

The balance between different regions of segments of the heart as well as the upper and lower tension forces creates event markers and motion patterns for both local and global activities under all phases of the cardiac cycle. These activities that can be registered from several points/regions both inside and outside the heart, can form a robust basis for assessment and classification of both the heart's local and global functions

The above review has described the fundamental anatomical conditions that need to be fulfilled in order for the heart to be described as a pump built according to the DAPP-technology

This review will now be supplemented with a theoretically established CSD to highlight how the heart's local and global hydromechanics/dynamics affects each other over time.

Description of the cardiac cycle phases as defined in the CSD and their related event markers and patterns

### SUMMARY

With the DAPP-technology defining the heart's pumping and regulating functions, it is possible to define and find relations between motion and balancing forces inside and/or outside the heart to define, validate, differentiate and classify local and global mechanical functions from each other. This will give many opportunities to find event markers that are depicting not only local mechanical performances of the heart but also summarized global performance of the heart described as CSD. Of particular interest is the event markers associated to the mechanical activities of the AV-piston.

The heart's, and thereby also the AV-piston's, possibilities to move by sliding inside the pericardial sack and furthermore the pericardial sack's possibilities to under certain conditions move and change its form in relation to its surroundings gives in both theory and practice good possibilities to find segments/regions, both within or outside the pericardial sack, that in balance with each other facilitate the optimal movement pattern and function of the AV-piston as well as compensating to uphold continuous inflow to the heart and low filling pressures.

These balanced or non-balanced motions can on both local/regional and global level be found in the form of event markers in time varying signals and/or motion patterns that reflect both the heart's local hydromechanics and global hydromechanics/dynamics.

A theoretically established complete CSD and its underlying mechanical background can constitute basis for an organized timing and pattern recognition framework connected to reference databases to e.g. support heart and circulatory diagnostics. It can furthermore constitute a basis for how to optimize placement of measurement regions or points of measurements (ROI), to obtain rich signals that reflect e.g. balanced time markers and motion patterns even from rather simple monitoring equipment and investigation methods.

Detailed Description of the Cardiac Cycle Phases

The Atrial Contraction and its Resultant Effects on the AV-Piston

By contraction of the atrial musculature in atrial systole, the atria and its wedge shaped auricular appendages that are situated in between the pericardium's outer egg-shape and the hemispherical peripheral segments of the AV-plane, will be pulled away from the AV-plane towards the centre of the atrial volumes. By the hydraulic fixation of the auricular appendages in between the AV-plane and the pericardium, the AV-plane will upon retraction of the auricles actively be displaced from its natural resting position and pulled up towards the basal plane of the heart.

Concurrently to this displacement of the AV-piston in basal direction, the ventricular musculature will be stretched out and there will be a redistribution of blood volume between the atria and ventricles. Furthermore, basal displacement of the AV-piston, will increase its peripheral ΔV-areas (FIG. 1b) as well as pull its central ΔV-areas toward the basal plane of the heart resulting in a volume expansion created by displacement of the central ΔV-areas, resulting in a ventricular volume increase, central ΔV-volumes, that must be filled. Filling of these volumes can happen through either a peripheral shrinking of the pericardium, i.e. reducing the total volume of the heart, and or by increased inflow to the heart. The latter filling mechanism prevents backflow out from the atria during atrial systole.

The greater work that the atrial contractions need to perform to be able to pull the AV-piston toward the heart's basal plane, as in e.g. increased stiffness of the ventricular musculature, the greater risk is there that this work will give rise to a backflow out from the atria which disturbs the dynamics of the heart's inflow.

At low frequencies and flows, there can under normal conditions be small backflows out from the atria which can easily be absorbed by the compliance volumes in the filling vessels (vena cava and pulmonary veins).

The displacement of the AV-piston towards the heart's basal plane and the re-distribution of blood volume occur with open valves and a relaxed IVS. This normally occurs without or with very low pressure gradients across the AV-piston. This means in principle that the purpose of the atrial contractions primarily is to stretch out the ventricular musculature and to displace the AV-piston, its outflow vessels and their blood contents in direction of the base of the heart.

Since there are no pressure gradients generated across the AV-piston, there can neither be any pressure gradients large enough to contribute to stabilizing any potential segmental differences in the atrial contractions effects on the AV-piston.

The local or regional net force exerted by the atrial musculature will depend on what resistance or counter force that the stretching out of the opposing region of the ventricular musculature creates.

Since there are no pressure gradients generated across the piston the internal tension forces in the ventricular musculature cannot in the same manner as under pressure equalize deviating tension within different segments of the ventricular musculature.

Since the movement of the AV-plane toward the heart's base plane in atrial systole will not only be affected by the actions of the atrial musculature, it could also reflect if there are any regional or segmental differences in the elasticity of the ventricular myocardium.

This time interval is thus well situated for finding event markers and or movement patterns of e.g. the AV-piston to find regional or segmental deviations in the ventricular myocardium's resistance to tension. It can also be a part of a State Index (SI) for further highlighting deviations in cardiac mechanical performance.

Pre-Ejection Phase

Earlier, the pre-ejection phase has not been clearly defined from a hydro mechanical viewpoint and thereby it's starting and end-points have also been ill defined. The phase has been called “isovolumetric phase” but this is an ill definition when considering the hydro mechanical properties of the entire time interval occurring between the end of the atrial contraction and the start of the ventricular ejection phase as it when it is correctly defined hydro mechanically involves a multi-functional interaction between incoming flow and volumes within the pericardial sack. This makes the term “pre-ejection phase” a more suitable universal name.

The heart is an elastic unit where the heart-musculature via contractile elements starts an entire series of interacting tensioned elastic components both within and outside the heart. The AV-piston's displacement toward the heart's basal plane during atrial systole means that internal tension forces will be formed in the ventricular musculature which thereby also affects the RsA. This tension means that the AV-piston passively, without any help from a started ventricular myocardial contraction, can start to move towards the apex and begin to close its inflow valves.

Thereby, event markers and motion patterns can in the beginning of this phase be seen that, just like the event markers during the atrial contraction phase, depicts segmental deviations in the tension forces of the ventricular musculature.

The initial ventricular contraction will by continuing to move the AV-piston toward apex add additional tension forces to the ventricular musculature. The AV-pistons peripheral muscular surfaces in conjunction with the continuation of the ventricular musculature will initially mediate force and pressure toward the central surface of the AV-piston, the fibrous skeleton, to tension the sail-like leaflets of its inflow valves. Initially this occurs under low pressure and can, via the mediating function of the IVS, like the atrial contractions, be considered to encompass all blood volume and muscle mass inside the pericardial sack with the result that this phase has different event markers and starting and end points than the classic description of the “isovolumetric phase”.

The AV-piston's displacement toward the apex means that upper and lower external deltaV-volumes and RsA-volumes with resulting tension forces begin to develop. Event markers and motion patterns during this phase reflects how the mechanical pressure stabilization of the cardiac structure occurs. In this context, event markers and motion patterns related to the IVS conveys vital information about the heart's global functions.

Increased tension and force development by the ventricular musculature confers that the entire AV-piston, that initially has a large total surface, is pulled towards apex. Increased pressure in the ventricles results in increasing tension of the inflow valves and that the ventricular septum (IVS) takes a systolic position and shape that will withhold the left ventricular pressure. This gives IVS double-regulating functions, see further blow. Gradually there are pressure gradients created that which are nearly sufficient to open the outflow valves. This point in time defines the end of the pre-ejection phase and the start of the ventricular systolic ejection.

It is only towards the end of this phase that there are pressure gradients formed across the AV-pistons connections to the right and left ventricle. Thereby the muscle cells logistic orientations in spiral and helix configurations may redistribute the power to the AV-pistons motions so that any deviations in the pulling force developed by the ventricular musculature are evened out.

During this period, which can be seen as a continuation of the relaxation period of the atria, there are opportunities to find event markers and/or motion patterns that can be used to analyse when and how different regions of the ventricular musculature reaches the state when the transmitted power generating the AV-piston's motions is equalized, i.e. balanced.

This period is well suited for analysis of regional/segmental influence on the heart's global hydromechanics/dynamics and can be a part in State Index calculations to further highlighting deviations in cardiac mechanical performance. By registering how the IVS interacts between the right and left ventricle it is possible to acquire a basic idea about the heart's global functions.

Systolic Ejection Phase

This phase is in principle the time interval where all the energy transmitted to the circulatory system is generated and it has therefore mostly been studied in the context that the ventricles displaces a volume by the heart muscle cell's contraction forces.

Also with the DAPP-technology as background it is the intensity and length of this phase that constitutes the basis for the heart's hydromechanics/dynamics. However, a substantial amount of cardiac investigations are not focused on measuring how well the heart is functioning but rather if there are any signs of a manifest or impending myocardial infarction. Such signs can initially be local, transient hypokinesia/akinesias that may progress to a permanent dysfunction when an infarction occurs.

The systolic ejection phase is forceful and produces large motions in up to 5 motion axes affected by outgoing pressure and flow which even with very advanced investigation methods makes it very hard to detect local akinesias.

Therefore, the systolic ejection phase in the CSD will essentially reflect the energy addition that the net forces from the right and left ventricle transfers to the circulatory systems. This energy addition is also essential for the previous and subsequent phases to maintain the heart's well known normal properties.

The first small volumes that exits the ventricles are involved in tensioning the vessel walls which, because of the small mass accelerated, does not need a very large coordinated muscle work. After the first tensioning, there is rapidly a need a coordinated larger muscle work to transfer energy for continuing to increase pressure and acceleration of a growing blood mass. This occurs practically during the first 20-40% of the AV-piston's systolic expulsion phase which means that the AV-piston will be affected by the following internal and external dynamic sequence of events:

1. Through the earlier described longitudinal shortening of the ventricular musculature's outer contour, the AV-piston's motions towards apex will displace both muscle mass and blood towards the centre of the ventricular volumes which confers that pressure is generated that shall accelerate blood through and out of the ventricles. At the same time the atrial volumes and the inflowing blood are subjected to suction forces. These are formed as a direct consequence of the AV-piston's motion away from the heart's base plane and as an indirect consequence of the increase in the atria's width that is created by the AV-piston's peripheral deltaV-areas (FIG. 1a-d). The expansion of the atrial volumes, which also decreases the AV-piston's peripheral deltaV-areas, gives rise to pressure gradients that adds energy to increase and uphold inflow to the atrial volumes. The increased pressure in the outgoing vessels confers that their radial tension increases and they are at the same time subjected to longitudinal stretching from the AV-piston's motions toward apex.

The above described forces, together with the forces needed to tension out the atrial musculature and to overcome friction forces, are according to the earlier descriptions denoted as upper external tension forces. These are mediated through the ventricular musculature's internal tension forces and sliding motions along the pericardium as well as the direct connection of the IVS to the RsA and the RsA-volumes and their resultant lower external tension forces that are needed for the AV-piston to be pulled towards apex (FIG. 1a, 2a-d).

During low friction between the ventricular volumes and the pericardium, the upper and lower external tension forces, will through the ventricular musculature's internal forces and motions along the pericardium, balance each other during the ventricular systolic phases, see further below.

2. The AV-piston both determines the shape of the pericardial sack as well as slide inside it during its decent toward the apex. This means that the AV-piston during its motions toward apex gets an increasingly smaller circumference, i.e. the equatorial line gets an increasingly smaller circumference and smaller peripheral deltaV-areas along with expansion of the atrial volumes (FIG. 1a-d). The reduction of the AV-piston's peripheral surfaces fits well with that the heart's contractile forces as early as after 20-40% of the systolic ejection phase starts to decrease in intensity. The reduction of the AV-piston's surface does among other things make it possible for the ventricles to sustain the systolic ventricular pressures needed to maintain flow through the outflow vessels even though its net power starts to decrease.

The peak inflow to the atria normally occurs later than the peak outflow from the ventricles. This is more marked for the left side of the heart, depending on which capacity the atria's inflows have to fill out the expanding atrial volumes that are indirectly formed via the peripheral deltaV-volume's creation as described earlier.

3. Decreased upper external tension forces as results of a successive filling of the atria expansion as well as lower tension in the outgoing vessels as a cause of decreased systolic pressures also results in that the need for the lower counteracting balanced tension forces decreases. This means that the AV-piston gets better possibilities to move toward the apex instead of the other way around, which in turn improves the possibilities to sustain inflow to the atria.

When the power and tension forces in the ventricular musculature becomes too weak to sustain any outflow the Post-ejection phase starts

Regional hydromechanical alterations that under previous phases can visualize local alterations in the AV-piston's movements will during this phase, through the pressurization of the AV-piston and the ventricular musculature's elastic components, be evened out. However, the different regions will in the form of time- and motion patterns reflect how they are interacting to, as net forces, pull the AV-piston towards RsA.

These time- and motion patterns can be classified and compared with previous phases and the following phases to constitute a solid basis to identify local/regional hydromechanical functions. With ROI that visualize how the upper and lower tension forces are affecting the heart's surroundings as well as the motions of the IVS these ROI can provide fundamental information about the heart's global hydromechanical and hydrodynamic functions.

The heart's global functions can also be reflected in information related to the heart's inflow and outflow vessels. The global classification can further be based on classification of local/regional event markers in time and motion patterns.

Post Ejection Phase

The post-ejection phase starts when there is no longer any outflow from the ventricles. It thus starts before the outflow valves have had time to close.

Upon further weakening of the contractile elements, this leads to, like the pre-ejection phase but in reverse order, that the tension forces gives way for the diastolic pressure and a light backflow may close the outflow valves.

As earlier described, the total tension forces in the ventricular musculature does, except from the tension forces needed to sustain pressure and flow, also need tension forces to balance the upper and lower counteracting external tension forces.

As long as the internal tension forces in the ventricular musculature are strong enough to balance the upper and lower external counteracting tension forces, the chamber volumes will, after closing of the outflow valves, more or less be comprised of a ventricular solid hydraulically attached to the pericardium that consist of the AV-piston, the ventricular musculature and the blood volume that they contain.

After the closing of the outflow valves, there is further reduction in the pressure in the outgoing vessels. This gives decreased tension forces and thereby decreased resistance for longitudinal stretching out. Also, any remaining kinetic energy in the flows into the atria will widen these so that the AV-piston's peripheral deltaV-areas can be completely extinguished (FIG. 1a-d). This leads to an increase in pressure toward the top side of the AV-piston. This increase in pressure in conjunction with reduced tension forces in the outflow vessels means that the lower external tension forces can be reduced. This can result in that the ventricular solid and the surrounding pericardium can start to move toward the RsA-volumes. Positioning of the ventricular solid in relation to the heart's base plane means that the motion of the whole solid towards the RsA-volumes will give space for further inflow of blood to the atria volumes.

The RsA-volumes that in principle reduces the AV-piston's potential stroke length can in the above described way be used to increase the distance between the AV-piston and the heart's base plane and make way for continued inflow to the atrial volumes despite that the AV-piston's actual movements toward the apex has come to a halt.

In this way, also the lower external energy storages can be used to uphold inflow to the atria during the time that is needed for the repolarization process to reach a stage where the fast-filling phase can begin.

When the internal tension forces in the ventricular musculature can no longer connect the remaining upper and lower external tension forces with each other this leads to that the inflow valves starts to open and the fast filling phase starts.

The local/regional hydromechanical functions during this phase can be seen as a direct continuation of the systolic ejection phases from the right and the left ventricle with the one difference that the combined tension forces in the ventricular musculature cannot displace volume to uphold any outflow. This phase normally starts earlier in the left ventricle.

The classification of the local/regional hydromechanical activities can e.g. during this phase be based upon event markers and pattern recognition related to the closing of the outflow valves.

As earlier described the pressurized AV-piston and the ventricular musculature's elastic components will make regional/sector muscular work to be evened out. At the end of the ejection phase and the beginning of this phase and especially at the end of this phase the intraventricular pressures will become low. Differences in regional performances will clearly show up again. Motion patterns and event markers, can during the post-ejection phase be classified and compared with previous phases to see how active or inactive a certain segment is. These local/regional motion patterns and event markers can further be linked to and classified in association to, the following fast filling phase.

The global functions and their classifications can be performed in similarity with the ones described during the systolic ejection phase.

The Fast Filling Phase as Well as the Return Motions of the AV-Piston

When the relaxation process has reached a level where the remaining upper and lower external tension forces can separate the contractile elements, all stored energy is released which leads to the fast filling phase and the AV-piston's returning motions.

The upper peripheral deltaV-volumes above the equatorial line, show that they except from generating a forced expansion of the atria also to a large extent encompasses the upper ventricular volumes (FIG. 1a-d, 2a-d).

The upper peripheral deltaV-volumes formed during ventricular systole creates in conjunction with the lower RsA-volumes upper and lower compliance volumes that encloses the heart except toward the thoracic wall and the heart's basal surface. When the ventricular musculature no longer can resist the upper and lower externally formed tension forces there are initially suction forces developed that can add energy and give room for continued flow into and through the heart.

Initially the upper and lower external tension forces are working together so that there is a forceful acceleration of inflow created in the motion direction that may have been started toward the RsA-volume during the post-systolic ejection phase. The inflow is rapidly directed toward refilling the centrally formed deltaV-volumes and to restore the peripheral deltaV-areas by refilling the upper and lower compliance volumes. Thereby pressure gradients are formed that repels the AV-piston from the apex. The AV-piston's re-established deltaV-areas will be subjected to the largest pressure gradients toward the heart's surroundings which mean that the AV-piston's return happens like a continuous stretching out of the ventricular musculature from the fibrous skeleton down toward apex. Thereby the peripheral deltaV-areas can be restored. The central DeltaV-areas remain more or less constant during the entire cardiac cycle

The central deltaV-volumes and the restoration of the peripheral deltaV-areas are associated to the AV-piston's returning motions.

The return of the AV-piston is associated to refilling of the central deltaV-volumes and the restoration of the peripheral deltaV-areas. Thereby the AV-piston has a hydro mechanically inflow-controlled return.

The speed of the AV-piston's return will be much dependant on how large the deltaV-areas and the upper and lower compliance volumes are in relation to the heart's inflow. Nature has equipped the AV-piston with central, firm and peripheral adaptable deltaV-areas, where the latter under both ventricular systole and diastole are dependent on the heart's inflow. The AV-piston's peripheral deltaV-areas have in the end of ventricular systole decreased by a reduced circumference of the equatorial line and widening of the atria and their expansion out over the AV-piston's rounded, peripheral surface. Thereby there are, with need for smaller inflows, possibilities for the AV-piston to return to its starting position within the constraints of a thinner egg-like shape of the pericardial sack with full utilization of the tension forces in the outgoing vessels.

By the AV-piston returning like a piston with smaller deltaV-areas, its return can happen more rapidly and at the same time there is space saved within the pericardial sack to allow continued filling of remaining upper and lower compliance volumes around the pericardial sack. This filling becomes more dependent on dynamic and static filling pressures in the heart's inflows.

The repelling of the AV-piston and apex from each other becomes both faster and more intensive during higher flows and frequencies depending on that the remaining upper and lower external tension forces as well as the kinetic energies in the flow through and into the heart are considerably more forceful than during lower flows and frequencies, see further below.

During the AV-piston's returning motions there is also a redistribution of blood between the atrial and ventricular volumes. That is, the AV-piston's return will divide the total volume inside the pericardial sack into smaller atrial volumes and larger ventricular volumes.

Local/regional deviations in the ventricular musculature toward the end of the post-systolic phase will be further confirmed if there is continued deviation in timing and motion patterns during this phase.

Local/regional differences in timing and motion patterns during this phase will further confirm if there are any local hydromechanical deviations.

The fast filling phase starts the time interval of the cardiac cycle that through the IVS in principle can be regarded as one large, single volume that consist of a more or less shiftable muscle mass and a blood mass enclosed in the pericardial sack.

During this period also the IVS takes a passive role which means that the AV-piston and the heart as a whole can be regarded as one single pump that is controlled by the DAPP-technology.

By placing ROI that can detect the motion pattern created by the upper and lower external tension forces and if possible also the motion pattern of the ventricular septum, objective timing information can be obtained that indirectly shows if the net forces from the right and left ventricle provides optimal hydrodynamic and auto-regulating functions. Also local hydromechanical function of the right and left atrium and ventricle can be visualized in this manner.

To improve the possibilities of presenting this information with robust signals, monitoring equipment and/or investigation methods can be adapted so that they manually and/or automatically, e.g. by coordinate functions, localizes ROI that optimally visualizes that heart's local/regional and global functions.

The Slow Filling Phase—the Resting Position of the AV-Plane

In association with and especially after the acceleration of the inflows to the heart during the fast filling phase the volumes inside the pericardial sack forms one large single compliance volume where the relaxed ventricular septum divides the ventricular volumes. This means that the AV-piston and thereby also the pericardium can be widened, regain peripheral deltaV-areas and take a resting form and position that is adapted to the actual inflow to the heart.

The position of the AV-piston and consequently also the position of the equatorial line is determined by the balance provided by the pressure gradients acting on to the restored peripheral DeltaV-areas.

During both the fast and the slow filling phase there is except inflow to the heart also redistribution of blood between atria and ventricles. The latter occurs when the AV-piston via forces acting onto the central and peripheral DeltaV-areas are forcing the AV-piston towards the base of the heart. Thus the AV-piston, with its large inlet valves, will slide over the inflow to the ventricles like a cylinder sliding over a “column” of inflowing blood.

During this phase, the global course of events can be detected by time markers and motion patterns that reflect the continued expansion and changes in shape of the pericardium, as well as the motion pattern of the IVS.

The Double-Regulating Functions of the Ventricular Septum.

As earlier described, the heart's four cavities will, in connection to and after the fast filling phase, be joined into one large, single volume enclosed by the pericardial sack and its external upper and lower compliance volumes, where the relaxed ventricular septum (IVS) by its movements may indirectly transmit both filling pressure and flow between the ventricles. Thereby the AV-piston's shape and motions will be affected by inflow both to the right and left side of the heart.

The ventricular septum is under normal conditions subjected to higher ventricular systolic pressure from the left ventricle than from the right ventricle (the inverse relationship is present during the fetal development). This means that the ventricular septum (IVS), despite of its shape and position in diastole, during systole under normal conditions, seen from a short-axis view attain a close to circular cross section.

If the shape and position of the ventricular septum during the ventricular diastolic time interval deviates from the shapes and positions that are formed during the ventricular contraction's pre-systolic phase, it may lead to that the IVS indirectly transfers a volume from one chamber to the other. This means that the motions of the IVS have two-way, double-regulating functions.

The Influence of the Outgoing Vessels on the RsA.

The outgoing vessels attached to the AV-piston have their outflow regions in close proximity to the IVS that separates the ventricles. During the pulling of the AV-piston toward apex, the walls of the outgoing vessels will develop resistance as they together with the AV-piston are pulled toward the apex. Their angled (T. Pulmonalis) and screw-like shapes (Aorta) can reduce the tensioning needed by the heart making a slight mechanical rotation inside the pericardium. The resistance that are created in the outgoing vessels longitudinal motions and tensioning are mediated mostly by IVS, supported by the Trabecula Septomarginalis, onto the diaphragm and further to the pericardial sack's postero-lateral limitations toward the surroundings.

This pulling as well as the right ventricle possibly having a larger peripheral deltaV-area and thereby develops more force toward its surroundings can be the causes that a slight, angled displacement of the left ventricle into the right ventricle takes place.

This slight displacement can be the cause for that the IVS under normal conditions attains a stable central position during the ventricular contraction while the left ventricle's posterolateral limits can be seen to create external volume displacements as a part of the RsA-volumes formed (FIG. 1a-d, 2a-d).

Open Cardiothoracic Surgery Damages the AV-Piston's Sliding Motions

It has been shown that open cardiothoracic surgery results in external friction forces and/or connective tissue adherences that greatly inhibits the AV-piston's motions and especially the motions of the outgoing vessels toward the apex.

This confers a forceful pull of the RsA up toward the heart's base plane with markedly increased displacement of the left ventricle into the right ventricle (which is not to be confused with the double-regulating functions of the IVS) which leads to large increase of the RsA-volumes and a large decrease of the DeltaV-volumes.

The resistance to pull the AV-piston toward the apex and away from the heart's base plane will greatly reduce the stroke length of the AV-piston. This results in that no energy or room, both directly via diminished AV-piston motions and indirectly by greatly reduced upper peripheral and central deltaV-volumes, are created to uphold inflow to the atria during the ventricular systolic time interval. The large reduction of the external compliance volumes surrounding the atria results in a discontinued inflow to the atria and an increased need for inflow pressure. If the atrial appendages are incised and ligated, which is frequently performed in cardiothoracic surgery, the hydro mechanical conditions will become even worse.

The stored energy during ventricular systole that is associated to externally formed volumes and normally adds energy to the inflow both during systole and diastole is no more or less concentrated to add energy to the inflow during diastole and relief of the large RsA-volumes.

A large part of the heart's inflows will now happen during its diastolic period which in principle means that the heart has turned into an ordinary displacement pump which has several hydrodynamic drawbacks such as increased filling pressures, valves closing with backflow etc.

In studies of new cardiothoracic surgery techniques, where the AV-piston and its outflow vessels can continue to slide under low friction inside the pericardium, with preserved properties of the DAPP-technology, the heart mechanics normalize recover after only one or a few days and patients have a very speedy recovery (compared to months or even years with established techniques).

This shows an example of how purely external mechanical alterations in the form of increased resistance for the AV-piston and its outgoing vessels to move, dramatically changes the conditions necessary for the balancing functions of the AV-piston to sustain a dynamic flow into and through the heart.

Despite these very pervasive changes in the heart's dynamics, they cannot be observed through conventional investigation methods. These methods are mostly focused on analysing the contractility in different regions of the heart which can be entirely normal even if the RsA-surface is pulled toward the AV-piston instead of the other way around.

High flows and frequencies provide high kinetic energy levels into, through and out of the heart.

At high flows and frequencies there are high kinetic energies entering, going through as well as leaving the heart. These energies are added, just like at low flow and frequency, by the contractile elements influence on the ventricular musculature's elastic components.

These will, just as for low flow and frequency, in a similar but much more intense way, affect the upper and lower external tension forces and their associated deltaV- and RsA-volumes.

At high flow and frequencies the heart's fast filling phase will bridge the slow filling phase. The AV-piston has already during the fast filling phase theoretically hydrodynamic properties required to, attain an upper position of at least the central part of the AV-piston that can coincide or even exceed the uppermost position that is set by the atrial contractions under normal flows and frequencies. Furthermore there are theoretically hydrodynamic possibilities for the RsA to be pushed beyond its resting position.

This means that the distance between the AV-piston and the RsA-surface already at the end of the fast filling phase exceeds its normal neutral position, which results in a stretching out of the ventricular musculature. The tension forces within the ventricular musculature may further increase by a continuous inflow that can fill out remaining external complience volumes and create an expansion of the AV-piston during the atrial contraction time and the time it takes to initiate the ventricular contraction.

In this way the pericardium and thus also the AV-piston attain shapes and positions that results in a volume of the heart that is adapted to the current inflow and frequency. The dynamic and static forces acting on to the DeltaV- and RsA-areas inside the heart provide the ventricular muscle cells with pre-tension forces that optimize their contractile forces and shortening. Furthermore the fast filling phase and the underlying kinetic energies may create vortex motions behind the inflow valves that actively contribute to close these before onset of the ejection phase.

The initial pre-systolic phases can be shortened by the generated pre-tension forces and higher cardiac inotropy that by greater force generations have the possibilities to start the systolic ejection phase earlier.

During the ventricular systolic ejection phase the contractile elements generate greater forces that via greater internal tension forces pressurize, accelerate and displace large stroke volumes into expanded outflow vessels in a nearly halved ejection time. This results in that larger kinetic energies are transformed into the outflow vessels which means that the vessel's pulse wave-conducting functions may give rise to low end-systolic pressures and low remaining tension forces in the ventricular musculature. Therefore the ventricular volumes will be emptied to the greatest possible extent with low remaining end systolic blood volumes.

During the end of this phase, just like earlier described but with greater underlying forces, a return of the solid ventricular volume toward the RsA-volumes may occur with continued inflow to the atrial volumes as consequence.

Continued depolarization of the ventricular musculature finally results in a separation of the ventricular musculature's contractile elements and the release of the remaining upper and lower external tension forces.

This release gives rise to the same course of events which has earlier been described but under considerably higher dynamic energy levels flowing into- and through the heart. The high energy levels confer that the AV-piston and the respective RsA-surface repels from each other under an explosive-like filling and redistribution of blood within the heart. The fast filling phase gives the AV-piston's back and forth-going motions a sawtooth-like motion pattern, which sets high demands on that the upper and lower external volumes and their associated tension forces are in balance.

Local and global activities can during high flow and frequency be registered and evaluated as earlier described.

Part Two—a Pattern Recognition Framework

The cardiac state system comprises a processing unit that is configured to identify and classify up to six main phases (MP1-MP6) defined in accordance with the DAPP-technology, using information in the received input signal. In addition the main phases are constructed using algorithms from all identified regional activities (RA) within or outside the heart. These regional activities can further be divided in one or several sub region activity (SRA) and/or curve segments and identified and classified to interpret, evaluate and classify their influence on the global mechanical functions of the heart.

By the cardiac state system in combination with reference databases (RDBs) all main phases (MP1-MP6) in a cardiac state diagram (CSD) may be classified and typed to facilitate and enhance the formation of the CSD from distorted information.

Pilot studies have shown that forming a state index comprised of both the standard deviation of sub region activities (SRA) and the mean value of sub region activities have a very high sensitivity and specificity concerning ischemic heart decease (AUC 0.98).

Thereby, the classification of region activities (RA) in relation to CSD not only improves the formation of CSD, it also depicts the origin of any mechanical dysfunctions, see below, of the heart. This detailed information can be summarised as Global and/or Regional dynamic characteristics or factors of the heart (DF, RDF) to be used e.g. for diagnosis, prognosis and treatment.

In addition this information is also used to update and refine the reference database DAPP-RDB.

In accordance with one aspect of the present invention search tools are applied that includes pattern recognition search algorithms configured to interpret and classify the dynamics and/or the mechanics of a heart, based upon basic dynamical and/or mechanical relations, and by using information in reference databases (e.g. DAPP-RDBs) including both theoretical and authentic Cardiac State Diagrams (CSDs), and other relevant information.

The cardiac state system is configured, by using the information in DAPP-RDB, and by applying e.g. different algorithms, pattern recognition, matching systems and rule based systems, not only to divide the heart cycle into its main phases (MP1-MP6), but also to illustrate how e.g. the dynamic characteristics of the heart are influenced by specific muscle segments during a heart cycle, i.e. regional activities.

An overall platform, a so-called GrippingHeart Platform (GHP), includes the cardiac state system and the DAPP-technology, algorithms, and reference databases, DAPP-RDB. In addition the platform may include other relevant information databases, e.g. anatomical databases.

In the following a brief summary of the present invention is given.

Various detecting apparatuses may be used to gather input data representing different aspects of heart activity.

The input data is pre-processed and analysed in order to identify specific landmarks (LM). In particular, simple identifiable landmarks (SLM) are identified, which represent easily identified events of the heart cycle, e.g. peak segments of an ECG-curve.

The identified landmarks are used, to identify several points of interest (POI) and from these points are derived event markers in every time interval pointed out of every land marks intervals by applying DAPP-mechanics and -algorithms. These event markers are then used to establish the phases of a Cardiac State Diagram (CSD).

In some occasions all phases may be identified. More often, some main phases (MP) of the CSD are missing.

For each missing main phase a search procedure is applied. The activities from certain areas will according to the heart mechanics have impacts on to the global heart functions where information available in a reference database (RDB) is used to identify the missing phase. More specifically, the present heart rate, age, gender, and other relevant information from the patient can also be used when accessing and searching/matching e.g. curve segment from specific region activities (RA) in the reference database (RDB). The search is often iterative, and initially the RDB will come up with a suggested curve-form in a global and or local activity form based upon secured basic CSD-data and relevant patient-related information.

The suggested curve-form is compared by using pattern recognition/matching or other relevant algorithm and basic mechanics to the detected curve in order to identify similar curve portions. The search is repeated until all main phases have been identified. If not all main phases can be identified, i.e. one or many phases are missing the system will display only the correct identified phases.

The present invention will now be described with references to the figures.

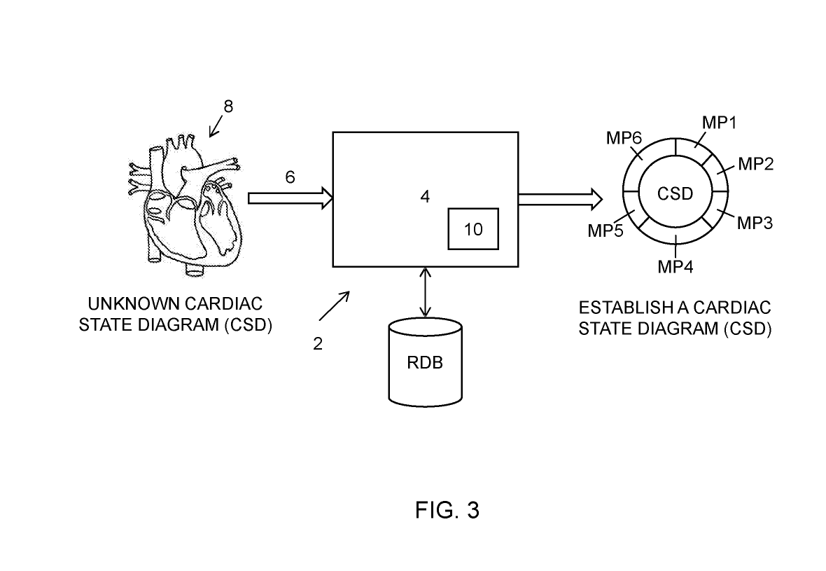

First, with references to the schematic block diagram illustrated in FIG. 3, the cardiac state system 2 will be described. The cardiac state system comprises a processing unit 4 configured to receive input signals 6 including parameters from, or related to (e.g. simulated data), one or many registration points or areas within or outside a heart 8. These parameters preferably include one or many of acceleration, velocity, positions, etc. measured by advanced investigation methods where the heart functions are presented as complex series of images, e.g. ultrasound, computer tomography, or MM, and/or from less advanced investigation methods, e.g. pressure- and flow-sensors, accelerometers, or radar sensors.

The processing unit 4 is realized by one or many computers having sufficient processing capabilities of handling large amount of data. The cardiac state system further comprises a storage unit 10, e.g. arranged within the processing unit 4, where one or many search tools are stored. The search tools include various computing tools, such as one or many pattern recognition rules.

The processing unit 4 is configured to process the input signals 6, by applying the search tools, to identify point of interests (POI), being landmarks, patterns and/or group patterns, and also e.g. derived patterns and/or derived group patterns.

The POIs are classified according to a rule based model of how different tissue and/or hydro mechanical forces in the heart and circulatory system interact, to evaluate hydro-mechanical and/or hydro-dynamic functions of the heart.

Furthermore, the processing unit 4 is configured to search for and identify global and/or regional event markers among the POIs to evaluate hydro-mechanical and/or hydro-dynamic functions of the heart. Preferably, at least some of the identified event markers are associated to the AV-piston defined according to the dynamic adaptive piston pump (DAPP) technology.

The expression event marker should be broadly interpreted to include any point or group of points, or other similar representations, representing relevant positions, movements, velocities, accelerations, etc.

The search tools include various processing tools, e.g. mathematical functions, pattern recognition/matching systems, search algorithms, comparison rules, etc.

The POIs preferably includes simple landmarks (SLM), being easily identifiable characteristics of the input signals 6 representing easily identifiable heart events.

According to one embodiment the search tool comprises a search tool configured to search for event markers associated to the AV-piston.

In another embodiment the search tool comprises a search tool configured to search for counteracting event markers, and that at least some of the identified event markers are associated to counteracting forces between two or more points/areas that describe essentially the same event marker of the heart's hydro-mechanical and/or hydro-dynamical performances. The counteracting forces are preferably more or less opposite to each other.

According to one embodiment the processing unit 4 is further configured to identify at least one heart cycle and one or many main phases of six main phases (MP1-MP6) timely dividing said heart cycle, at least based upon the identified event markers. The six main phases (MP1-MP6) are defined in accordance with the DAPP technology and are used to establish a cardiac state diagram (CSD).

Advantageously, the processing unit 4 is configured to determine if all six main phases have been identified and if so a complete CSD is established.

Sometimes not all six main phases have been identified, in that case the processing unit 4 is configured to iteratively connect to a reference databases (RDB) to identify missing main phase or phases by applying the search tools. The reference database (RDB) includes classified data representing complete cardiac state diagrams (CSDs) including six main phases (MP1-MP6) and established in accordance to the DAPP-technology. The data in the reference database (RDB) is classified according to a predetermined classification scheme including one or many of age, gender, heart frequency, treatment data, e.g. heart frequency blood pressure treatments etc. and that the stored data includes curve forms representing the main phases.

The processing unit 4 is further configured to determine a so-called dynamic factor (DF), as a result from one or more region activities (RA), being a measure of the total pumping and controlling functions of the heart, for the presently determined CSD. According to one alternative the DF indicates a deviation in relation to a normal DF for a normal CSD determined during similar circumstances, and when more region activities (RA) are involved these activities can also be compared as local function parameters (LFP) both to the presently determined DF and to a normal DF for a normal CSD determined during similar circumstances. DF will be additionally discussed below.

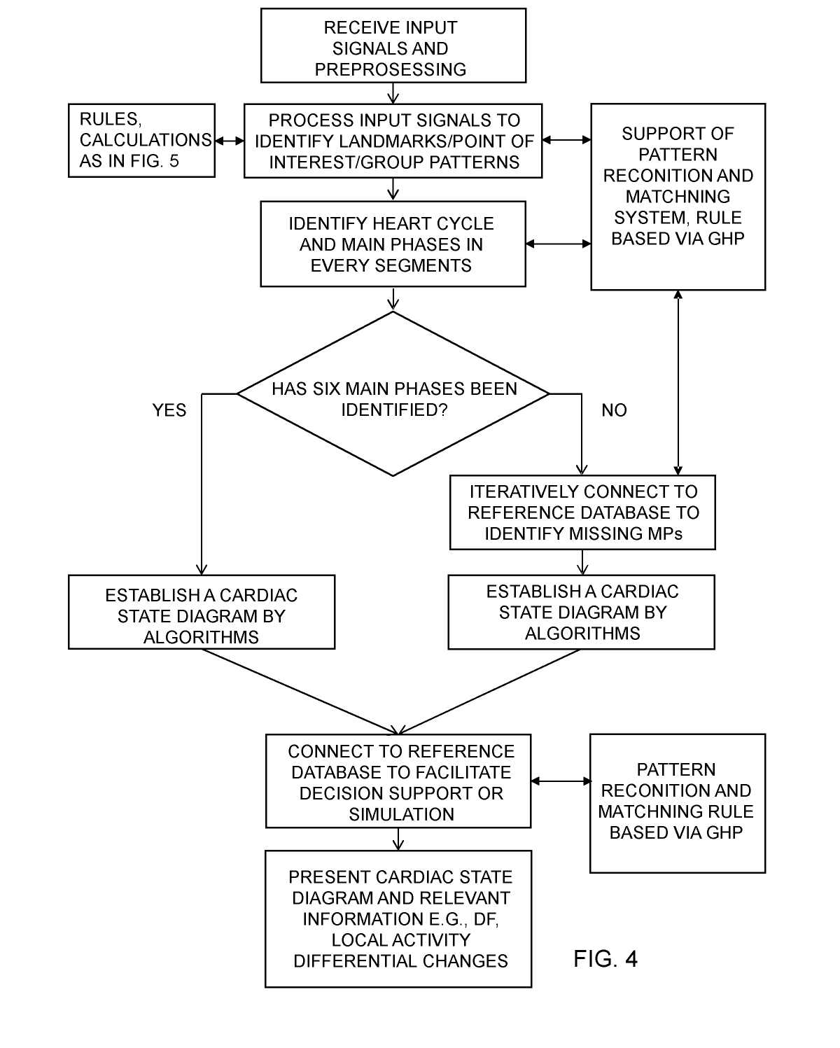

The method steps performed by one embodiment of the cardiac state system will now be discussed with references to the flow diagram in FIG. 4.

As described above the cardiac state system comprises a processing unit and a storage unit where one or many search tools are stored. The method comprises:
receiving, by the processing unit, input signals including parameters from, or related to, one or many registration points or areas within or outside a heart. These parameters have been exemplified above.

The method further comprises:
processing the input signals in said processing unit, by applying search tools, to identify point of interests (POI), being landmarks, patterns and/or group patterns,
searching for, and identifying global and/or regional event markers among the POIs to evaluate hydro-mechanical and/or hydro-dynamic functions of the heart, wherein at least some of said identified event markers are associated to the AV-piston defined according to the dynamic adaptive piston pump (DAPP) technology.

Preferably, the search tools comprise a search tool configured to search for event markers associated to the AV-piston.

In addition, or as a complement, the search tool comprises a search tool configured to search for counteracting event markers, and that at least some of the identified event markers are associated to counteracting forces between two or more points/areas that describe essentially the same event markers of the heart's hydro-mechanical and/or hydro-dynamical performances. The counteracting forces are more or less opposite to each other.

According to one embodiment the method comprises:
identifying, by the said processing unit, at least one heart cycle and one or many main phases of six main phases (MP1-MP6) timely dividing said heart cycle, at least based upon event markers. The six main phases (MP1-MP6) are defined in accordance with the DAPP technology and are used to establish a cardiac state diagram (CSD).

Furthermore, if it is determined, by the processing unit, that all six main phases have been identified a complete CSD is established.

Alternatively, if not all six main phases have been identified, the method comprises:
iteratively connecting to a reference database (RDB) to identify missing main phase or phases by applying the search tools. The reference database (RDB) includes classified data representing complete cardiac state diagrams (CSDs) including six main phases (MP1-MP6) and established in accordance to the DAPP-technology.

The reference database (RDB) not only includes classified data representing complete cardiac state diagrams (CSDs) with six main phases (MP1-MP6) and dynamic factors (DF), but also includes curve segment activities data and classification in different region activities (RA) established in accordance to the AV-piston motion and its effects to the heart, it's surroundings, in- and outflow, established in the GrippingHeart Platform (GHP).

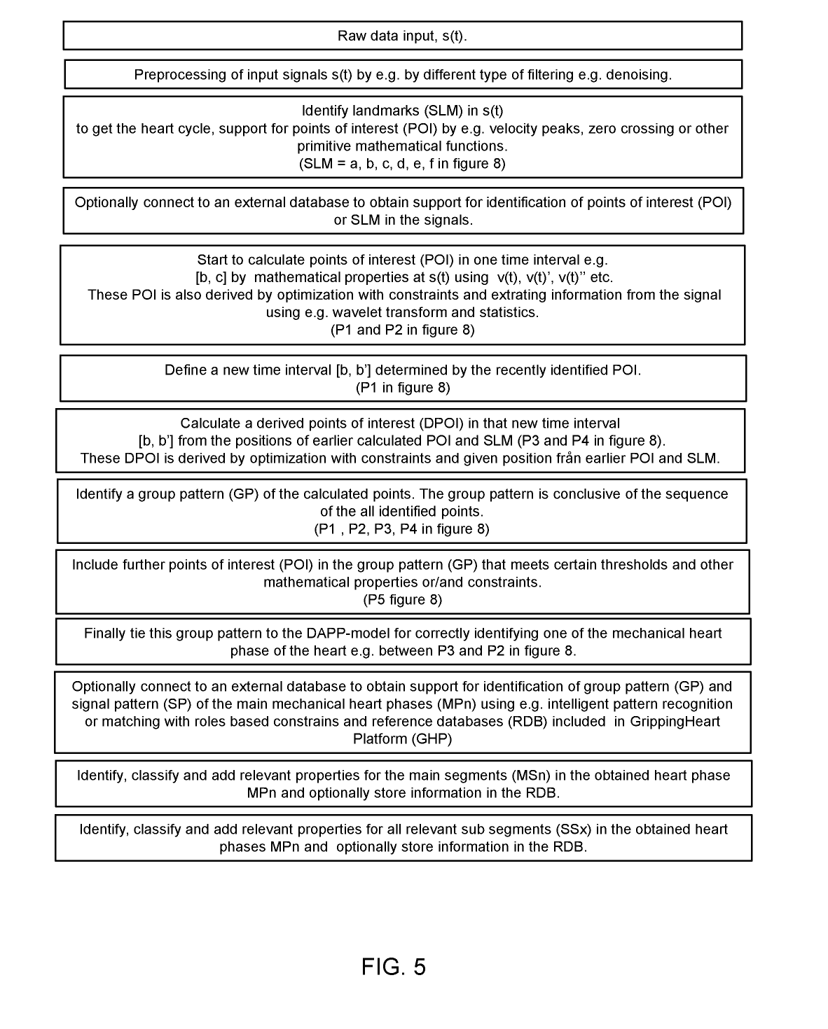

FIG. 5 shows a more detailed flow diagram of how to process the raw input data of the input signal in order to determine points of interest and event markers. The flow diagram can be combined with pattern recognition or/and matching for more accurate detection of all events or/and sub activities. In the flow diagram it is referred to the signal curves illustrated in FIG. 8.

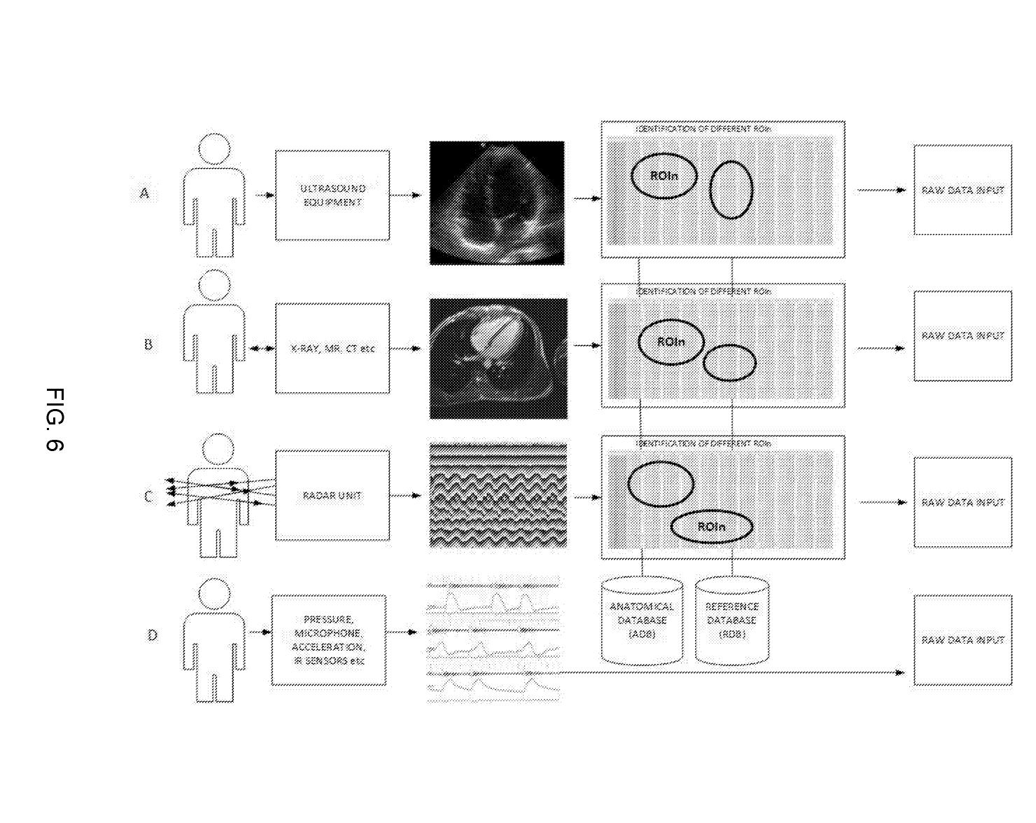

With references to FIGS. 6-13, various aspects of the present invention will be further highlighted.

FIG. 6 illustrates examples of detection methods (denoted A-D) for detecting different movement patterns of the heart. The examples are illustrated by an image of the heart or by signals representing various parameters, e.g. pressure, accelerations, etc. and different Regions of Interest (ROI) are then identified. As a result of the measurements a set of raw data input is obtained, being the input signal for the sub-sequent steps. During the identification of ROIs, and during sub-sequent steps, different databases may be used, e.g. anatomic databases or reference databases (RDB).

In A, ultrasound equipment is used.

In B, X-ray, MR or CT equipment is used for the detection.

In C, a radar unit is used.

In D, a pressure sensor, a microphone, an acceleration sensor or an IR sensor is used.

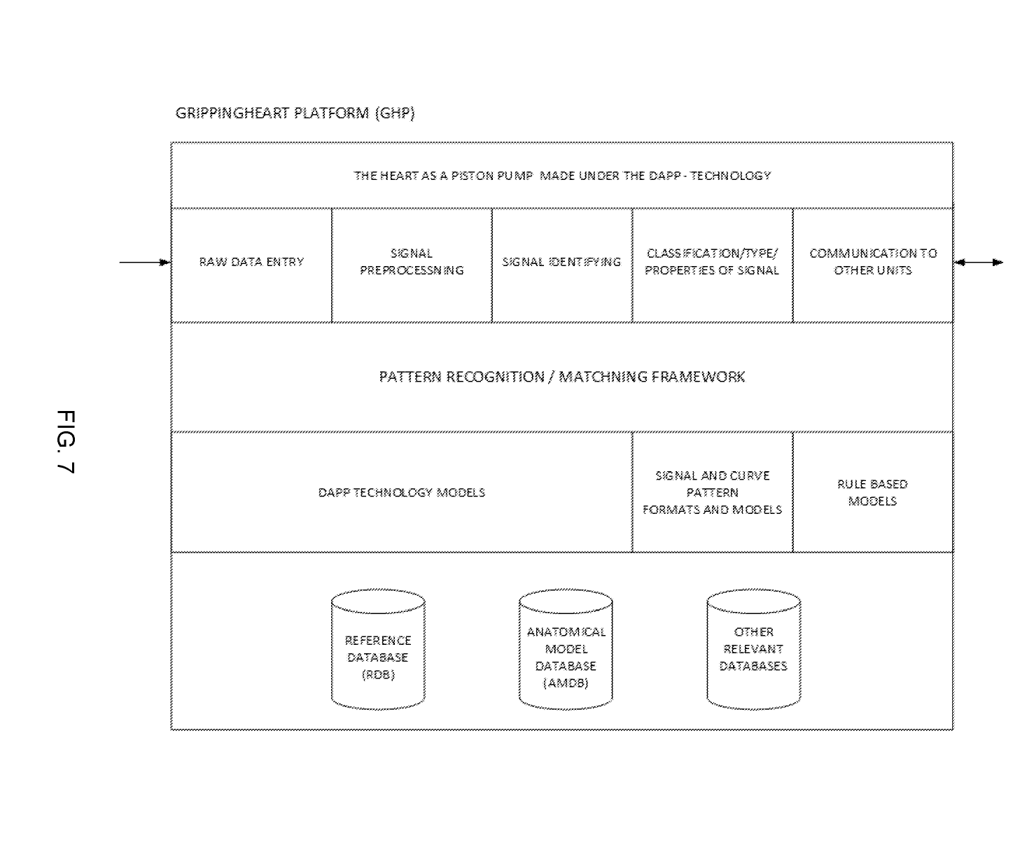

FIG. 7 is a schematic high level illustration of the cardiac state system, including the so-called GrippingHeart Platform (GHP), according to the present invention.

According to the high level description of FIG. 7 the system is configured to treat and process input signals, in the form of raw data entry, in order to establish, validate and analyse characteristics and functions of the heart. This is e.g. based upon theoretical and/or authentic signal patterns from reference databases and other relevant databases. In the figure various functional blocks are shown, which are briefly described in the following.
A pre-processing system configured to e.g. filter the input signals.
A signal identifying system which is used during the pattern recognition procedure.
A classification and typing system, which is used for classifying the identified phases and also to denote a type and properties to the classified phase.
A communication block for performing various communications to external units.
Databases, e.g. algorithm and pattern reference databases, and anatomical and other databases.
Models.
Algorithms, and rules.

In FIG. 8 is shown raw data entry examples in the form of input signals from a region. Herein it is also referred to the rules set forth in FIG. 5. The signal represents measured velocity and acceleration. In a first step the signal are post processed for e.g. noising, frequency/wavelets analysed in order to identify simple landmarks, SLM and/or significant patterns such that the cardiac state system will be able to establish a base level for continuous analysis and pattern recognition and to be a base for identifying point of interest, group pattern and/or pattern.

In the figure the SLMs are designated by circles.

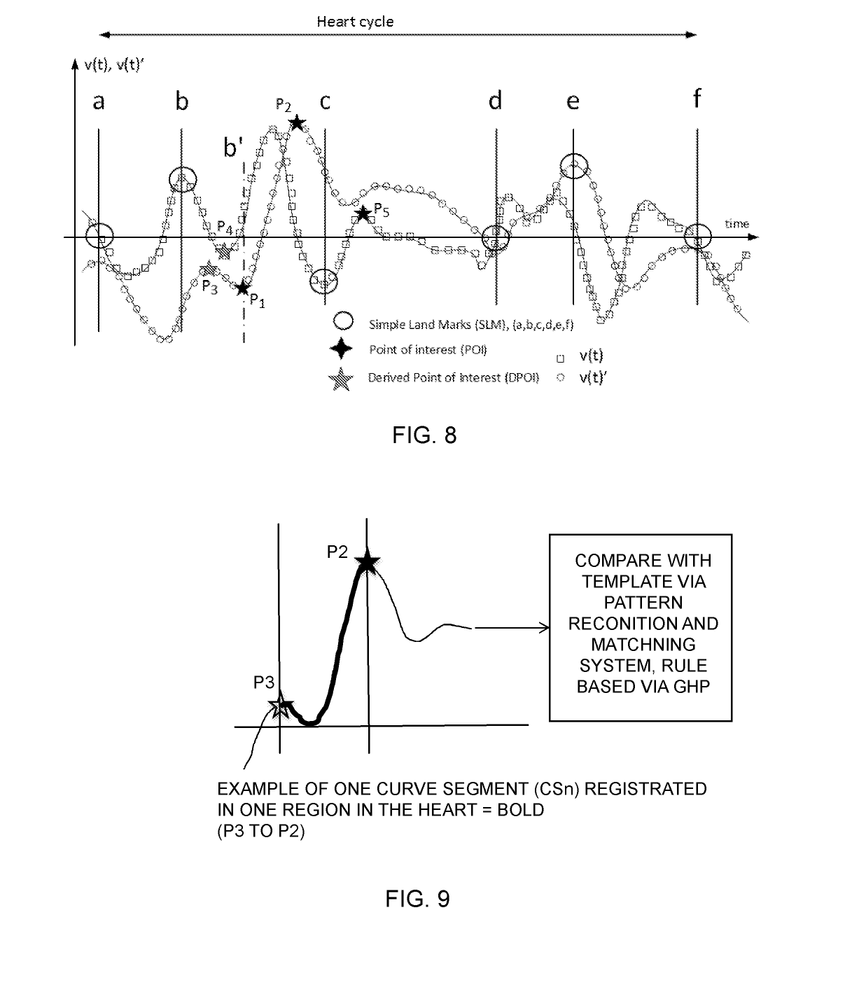

The next step is to analyse the pattern and/or signal by identifying points of interest (POI) and the derived point of interest and/or pattern (see FIG. 9) using the Gripping Heart Platform (GHP). From the different identified phases one or several curve segment (CSn) can also be identified and to be classified and optionally stored in the reference database (RDB). The point of interest is designated by a star in FIG. 9. These identified phases in the different input signals then form basis for establishing the cardiac state diagram (CSD) by using different algorithms and/or RDB (MP1-MP6). These may be used, at a local level, to determine how a specific point influences the global functions of the heart.

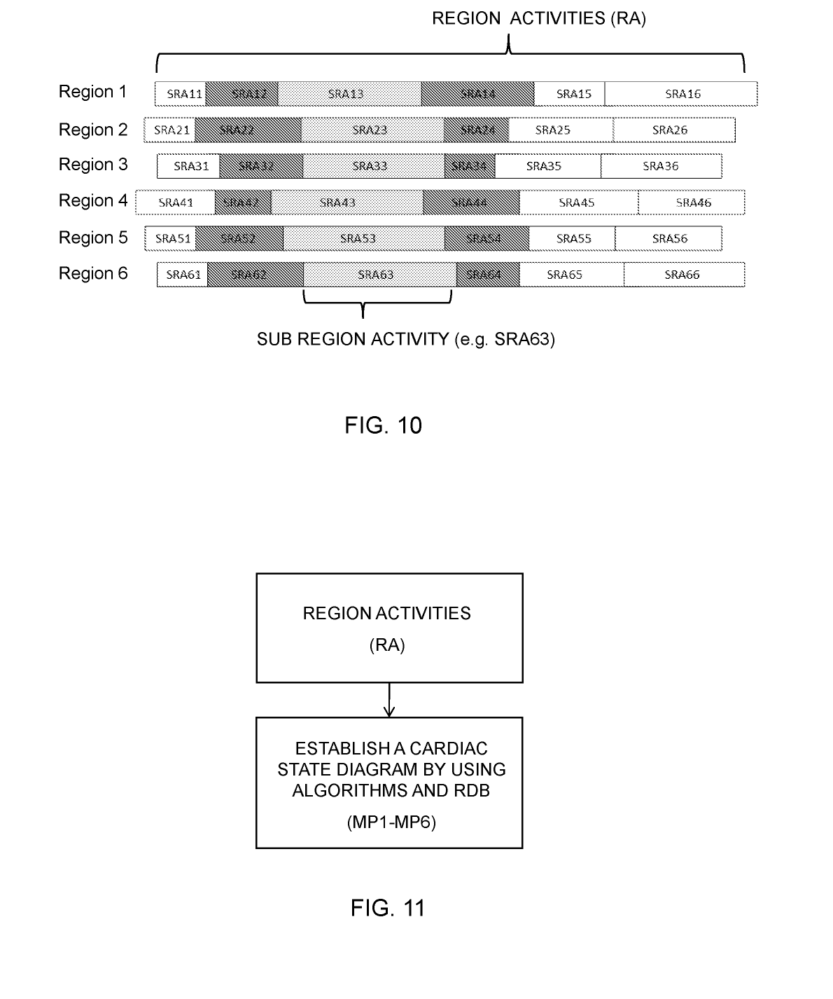

In FIG. 10 is shown an example of several region activities (RA) from several input regions, regions 1-6. By using the regional activities from these regions, a number of different indicators may be calculated; e.g. the state index, mean values and standard deviations for specific regions, etc. Below is one examples of calculating a State Index (SI) based upon the mean value SRA_MEAN and the deviation value SRA_SD to be e.g. used for taking clinical decisions.

Example of Calculating a State Index:

SRA_MEAN=MEAN(SRA12,SRA22,SRA32,SRA42,SRA52,SRA62)

SRA_SD=SD(SRA12,SRA22,SRA32,SRA42,SRA52,SRA62)

STATE INDEX=SRA_MEAN×SRA_SD

More specifically, the signal patterns of main phases are identified, classified, and typed by the Gripping Heart Platform (GHP).

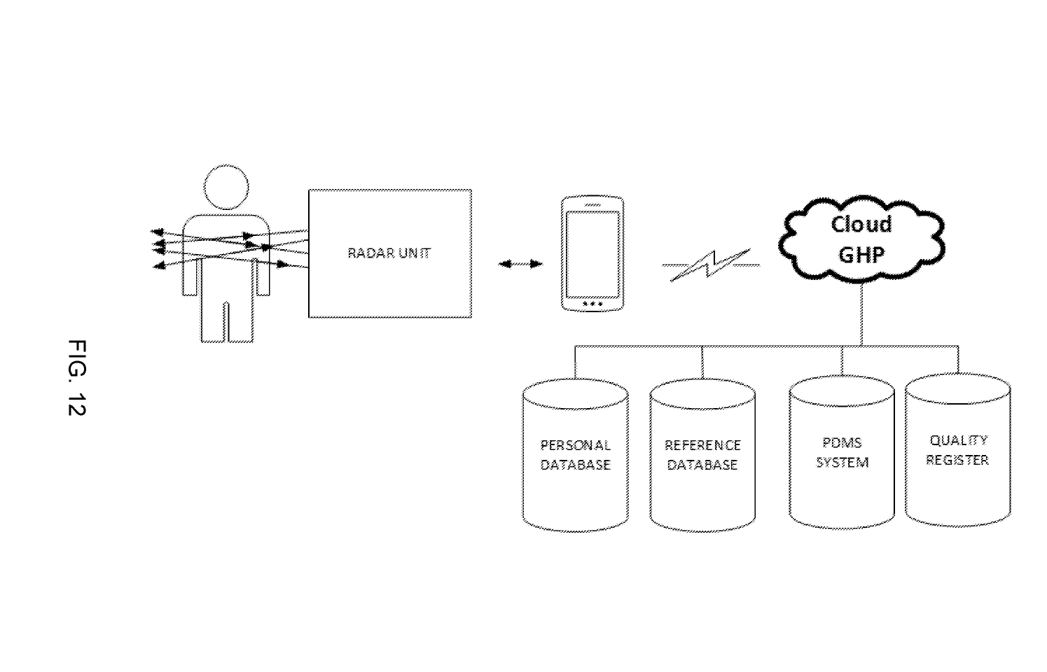

As a further step an identified main phase is typed, e.g. with regard to heart rhythm type (normal, sinus rhythm), that in turn is denoted further characteristics (e.g. amplitude, velocity, duration, acceleration). This information is essential for further evaluation and analysis. FIG. 11 shows the steps to establish the cardiac state diagram.

FIG. 12 shows a schematic illustration of one embodiment where the measurements are made by a small radar sensor unit. The radar sensor unit is provided with at least one antenna, preferably two or more. The number of antennas is dependent e.g. upon the level of accuracy required in the specific measurement. In one advantageous example the number of antennas was in the range of 5-10 antennas.

The heart movements are measured by a small radar sensor unit that may communicate, via e.g. a mobile phone or the communication cloud, with the cardiac state system and the relevant databases. Thereby a CSD including the region activities (RA) may be established that may be used as a fast and simple basis for analysis and diagnosis.

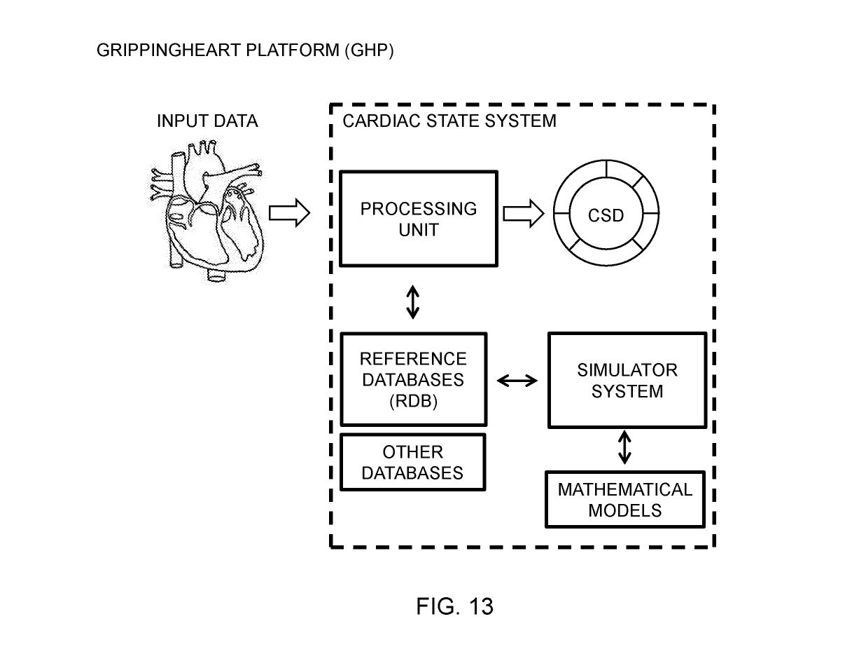

FIG. 13 shows how the Cardiac State System may interact with other systems in the GrippingHeart Platform to e.g. simulate the effects of different therapeutic interventions e.g. pharmaceutical, surgical, lifestyle or wellness. According to this embodiment the cardiac state system comprises a simulator system configured to handle a virtual heart and circulatory system and process virtual POIs that are classified according to a rule based model, e.g. based on the DAPP-technology, of how different tissue and/or hydro mechanical forces in the heart and circulatory system interact.

The purpose is to evaluate hydro-mechanical and/or hydro-dynamic functions of the heart in order to modulate and simulate what impacts different kinds of chemical, electrical or hydromechanical/dynamical parameters and other heart related information have to the rule based hydromechanical classification system. The processing unit 4 is further configured to iteratively connect to a reference database (RDB) presenting and receiving data and other heart related information that may be classified as global and/or local events, patterns and/or group patterns with or without score indexes.

One important aspect of the present invention is that each main phase should occur essentially at the same point of time irrespectively which registration points/area that has been chosen.

That is not the situation for the region signals for the single registration points/areas, instead they create a region pattern having a region activity (RA) illustrating the contribution to the time-related pump and control-function of the heart from that single point/area.

The input signals and their segmentation may be compared to both theoretical and authentic events and movement patterns stored in the reference database (RDB). The comparisons may be performed both prior, during and/or after the segmentation procedure.

The registration points are chosen such that they receive power and energy from a large number of muscle cells, e.g. from areas around where the AV-piston is attached to the annulus fibrous skeleton, or from the hydraulic connections of the heart muscles to the apical diaphragmatic surface of the pericardia and its fixation to the diaphragm.

The reason for choosing those registration points is to obtain larger movement patterns showing larger group of muscles' interactions to perform the pumping and controlling functions of the heart.

If these clear and often pronounced movements, via the Main Phases (MP1-MP6), are synchronised with other movement patterns (RA) in other points/areas/region within or outside the pericardium and intraventricular septum (IVS), a linked pattern of movement will be achieved, that in great detail reflects the mechanics of the heart.

In other words, the important aspect of the present invention is the fact that we know that these pronounced movements occur essentially simultaneously and therefore it is possible to identify and collect the missing information in other points in the RDB.

In the following steps 1-4 it is disclosed one implementation disclosing how the information of input registration data is processed and interpreted by the cardiac state system, and by the method applying the cardiac state system.

Step 1

Receive input signals to be used for analysing the functions of the heart and the circulatory system.

Signals from one or many registration points/areas having one or more “Region of interest” (ROI) are manually or automatically identified by using e.g. edge searching algorithms, anatomic databases, reference databases, etc.

These signals have its origin in the mechanics of the heart and may reflect changes with regard to movements, pressures and flows, with or without support of ECG-information. The signals may also be e.g. sound, vibrations, and light variations having connections to the heart and circulatory system.

The sensors or detectors used to obtain these signals may be positioned inside, on, or outside the body surface and cover one or many measurement points or areas.

Step 2

The cardiac state system comprises a processing unit. The processing unit is configured to analyse the input signals, to communicate with the reference database (RDB), framework, and to generate a result of the analysis. The input signals include e.g. velocities, accelerations, movements, and dimensional changes. The processing unit comprises a storage unit where various search tools are stored, e.g. pattern search algorithms. By applying those search tools the processing unit initially analyses the input signals in order to identify one or many so-called landmarks and/or patterns. A simple landmark is a curve form or pattern that is easy to recognize and identify, e.g. specific parts of an ECG-curve, the QRS-complex, etc. The SLMs may be used to determine the heart frequency and thereby also the heart cycle length and to facilitate the recognition of the pattern and/or point of interest (POI).

Step 3

After the initial analysis where easily identified landmarks have been identified the processing unit has established basic information of the heart subjected to measurements. This basic information may include the heart frequency and other relevant information that may be used for further processing of the signals, using more specific algorithms and search rules, based upon both theoretical and practical pattern recognition.

Then also the point of interest (POI) or/and pattern, group-patterns may be decoded and classified. These classified POIs are then, by using rules and pattern recognition, used to establish event markers which are a foundation for establishing the common main phases (MP1-MP6). See e.g. FIGS. 8 and 9.

The input signals, for each registration point/area, are divided by the six region activities (RA) that are further divided in sub region activities (SRA). These sub region activities may be identified in one or several curve segments (CSn). See FIGS. 10 and 11. The curve-segments represent energy changes and may be classified and typed to establish reference databases.

Local and/or central reference databases (RDBs) may serve as a basis for establishing an individual diagnosis, prognosis, treatment and follow-up, and simulation of different functions of the heart.

A local reference database is a database established and stored in relation to the cardiac state system, whereas a central database is remotely accessed.

See FIG. 7.

The Cardiac State Diagram (CSD) and its sub activities segments from each registration region/point/area reflect how the dynamic processes develop in the specific point or area. It is possible, by pattern recognition, to follow and classify how this point/area generates and receives energy during a heart cycle

The CSD including its Main Phases (MPn) is analysed. If the result of the analysis identifies deviations from expected results, then also one, or many, curve segments may deviate from its or their expected result(s).

Then may pattern recognition of the curve-segments, performed by the processing unit, not only confirm that the event markers for the main phases are correctly defined, but also show which region or regions related to the heart's pumping and controlling mechanics that deviate from expected results.

Step 4

The cardiac state system may be used to further investigate different factors that influence the heart functions. These factors may be both internal factors (e.g. medications, vessel constrictions, heart attacks) and external factors (e.g. age, gender, physical shape).

Dynamic Factor (DF)

In order to facilitate a usable measure of the degree of efficiency of the heart as a whole a so-called Dynamic Factor (DF) is defined that may be determined.

In addition, Local Function Parameters (LFPs) for different sub-region activities (SRA) are determined which represent the degree of efficiency of the heart with regard to mechanical pumping and controlling in different areas.

These dynamic factors, the DF and LFPs, may be determined promptly and may serve as an easily understandable basis for establishing an individual diagnosis, prognosis, treatment and follow-up and simulation of the different functions of the heart.

In one implementation the cardiac state system is configured to rapidly generate a simple representation of the heart mechanics. In order to reduce the amount of data the following procedural steps are performed:

Identifying, classifying and typing complete (see alternative A below) or incomplete (see alternative B below) CSDs. This is achieved by determining a Dynamic Factor (DF) for the heart as a whole.

As alternative, or in addition, numerous Local Function Parameters (LFPs) are determined by the sub-region activities (SRA) within each main phase (MPn) from one or many registration points/areas within or in connection to the heart.

Thereby an easily and quickly accessible indication of the efficiency of the heart as a whole may be established by the DF, and where and when problems occur during its mechanical pumping and controlling work, by the LFPs.

Thereby is created two pattern recognition alternatives A and B, that both, e.g. by applying the dynamic factors DF and LFPs, may serve as basis for comparisons in relation to expected values.

Alternative A

This alternative facilitates identification of markers/events/characteristics in the input signal which may be used to establish complete CSDs, i.e. identify at a maximum 6+6 time markers (right+left heart half) that build up the main phases (MP1-MP6). The markers' expected number and discernibility in relation to the heart frequency are classified and typed, and the total pumping and controlling functions of the heart are described as a compound dynamic factor (DF) as deviations from normal dynamic factor (DF) during similar circumstances.

In addition, the curve sub-segments (CSn) which are determined from one or many points/areas may be described as local function parameters (LFPs), as deviations from expected normal local function parameters (LFPs) for the corresponding points/areas during similar circumstances. This analysis strengthens and facilitates the decision-making regarding diagnosis, prognosis, treatment and follow-up.

Alternative B

This alternative facilitates identification of markers/events/characteristics in the input signal which may be used to establish non-complete CSDs. Here the signals, from one or many points/areas, between one or many main phases which have been established, will be subject to pattern recognition by the processing unit to determine e.g. LFPn.

Thus, it will be possible to determine the dynamic functions of the heart without having all main phases of a heart cycle, by using simple registration sensors. This alternative is in particular useful for simpler registration alternatives for monitoring and follow-up of therapies and physical training.

Initially, and during the build-up phase and during continued research and development of the cardiac state system and the Gripping Heart Platform (GHP) a large amount of specific registration points/areas serve as basis for classification and typing of the main segments (MP1-MP6) and the curve sub-segments (CSn) in order to decode, by pattern recognition, the dynamic movement pattern of the heart.

The reason for the classification is to:
reduce the amount of data,
facilitate pattern recognition,
establish reference databases (local and/or central) that:
   gives supporting identification of main phases and pattern recognized sub-phases,
   may serve as templates for pattern recognition despite defected or corrupted input data (registrations) where one or many time markers, or even entire phases, are not present, to be able to nevertheless use the input data in order to establish a diagnosis or a therapy treatment.

Summary of part one and part two and background to the aforementioned claims.

In part one a model of the heart as a piston pump is described that points out how different tissue and/or hydro mechanical forces in the heart and circulatory system interact as well as how this interaction changes during the mechanical chain of events in the cardiac cycle It has further been described how the deltaV-areas of the heart's piston, according to the DAPP-technology, give rise to external tension forces that can give the heart a more continuous inflow, resulting in low filling pressures to the heart even at high frequencies. It has also been described how and when during the cardiac cycle different POI can be used to differentiate signals that represent global and/or local hydromechanical functions.

In part two, a pattern recognition framework describes how input signals can be processed classified and evaluated, where local hydromechanical functions can be evaluated as e.g. Local Function Parameters (LFP) and global heart functions with Dynamic Factors (DF). Part one and part two further supports the determining of State Indexes (SI) for further highlighting deviations in cardiac mechanical performance.

Together part one and part two forms a Cardiac State System. This system can as described above be used to decode, classify, evaluate and store data from input signals generated from different kinds of investigating modalities. It can further, especially through its iterative rule based classification linkage to reference databases RDB, be used for modulation and simulation of the heart's hydromechanics and its relation to the hearts in- and outflow. With a simulator system (FIG. 13) simulated input data and also questions that can be found in its well defined rule based databases (RDB), e.g. related to a just established CSD can be used to see and understand what impacts different kinds of chemical, electrical, hydromechanical/dynamical parameters and other heart related information have to local and global functions of the heart.

In this way the above declared invention not only is important to detect shortcomings in the hearts hydromechanical/dynamical performance but also to be used as decision support for pharmaceutical and surgical treatments, follow up of these and furthermore for fitness and training purposes.

The present invention is not limited to the above-described preferred embodiments. Various alternatives, modifications and equivalents may be used. Therefore, the above embodiments should not be taken as limiting the scope of the invention, which is defined by the appending claims.

1. A cardiac state system, comprising
a processing unit configured to receive input signals including parameters from, or related to, one or many registration points or areas within or outside a heart and that said input signals are obtained during a time length of at least one heart cycle, and a storage unit where one or many search tools are stored,
wherein the processing unit is configured to process the input signals, by applying said search tools, to identify points of interest (POI), wherein said POIs are classified according to a rule based model of how the interaction between different tissue and/or hydro mechanical forces in the heart and circulatory system changes during the mechanical chain of events in one heart cycle, to evaluate hydro-mechanical and/or hydro-dynamic functions of the heart, wherein the rule based model is an event and timing function rule based model.

2. The cardiac state system according to claim 2, wherein the rule based model is based on the dynamic adaptive piston pump (DAPP) technology.

3. The cardiac state system according to claim 1, wherein the processing unit uses search tool/tools configured to identify simple landmarks (SLM) in input signals from said POIs, representing easily identifiable heart events.

4. The cardiac state system according to claim 1, wherein said search tool/tools is further configured to identify at least one heart cycle and one or more main phases of six main phases (MP1-MP6) timely dividing said heart cycle to establish a cardiac state diagram (CSD).

5. The cardiac state system according to claim 1, wherein said search tool/tools is further configured to search for and identify global and/or regional event markers, patterns and/or group patterns among said POIs to evaluate hydro-mechanical and/or hydro-dynamic functions of the heart.

6. The cardiac state system according to claim 1, wherein said search tool/tools is further configured to search for event markers, patterns and/or group patterns associated to the motions of the AV-piston and/or the ventricular septum (IVS).

7. The cardiac state system according to claim 1, wherein the processing unit (4) is further configured, by using said rule based model, to search for and to analyse local/regional/segmental differences, from two or more registration points associated to the AV-piston motions, before and/or after tension forces within the heart-muscles have evened out any imbalances in the AV-piston's motion pattern.

8. The cardiac state system according to claim 1, wherein the processing unit is further configured, by using said rule based model, to search for and to analyse global hydromechanical/dynamical functions by input signals from one or more registration points outside the heart.

9. The cardiac state system according to claim 1, wherein the processing unit is further configured, by using said rule based model, to search for and to analyse global hydromechanical/dynamical functions of the heart by using two or more input signals, from registration points outside the heart, that more or less reflect counteracting forces that can be used to validate the timing and pattern of events.

10. The cardiac state system according to claim 1, wherein the processing unit is further configured, by using said rule based model, to search for and to analyse global/local hydromechanical/dynamical functions of the heart by using two or more input signals from external and internal registration points including registration points associated to IVS motions.

11. The cardiac state system according to claim 1, wherein if not all six main phases of a cardiac state diagram (CSD) have been identified, the rule based model and processing unit is further configured to iteratively connect to a reference database (RDB) to identify missing main phase or phases, wherein said reference database (RDB) includes classified data representing complete cardiac state diagrams (CSDs) with global and local event markers, patterns, group patterns and other heart related data and information.

12. The cardiac state system according to claim 1, wherein if all six main phases have been identified, the processing unit is further configured, by using said rule based model, to transfer global and local event markers, patterns and group patterns classified with or without score index according to a predetermined classification scheme and other heart related data and information to said reference database (RDB).

13. The cardiac state system according to claim 1, wherein said cardiac state system comprises a simulator system configured to compare a newly established CSD with previously classified CSDs and other heart related information stored in reference databases (RDB), in order to modulate and simulate what impact different kinds of chemical, electrical or hydromechanical/dynamical parameters and other heart related information have, to provide decision support when e.g. evaluating treatment options.

14. The cardiac state system according to claim 1, wherein said cardiac state system comprises a simulator system configured to apply mathematical models of the heart and circulatory system in order to modulate and simulate the impacts of different kinds of chemical, electrical or hydromechanical/dynamical parameters and other heart related information to provide decision support when e.g. evaluating treatment options.

15. The cardiac state system according to claim 1, wherein said input signals are obtained from at least one radar sensor unit provided with at least one antenna.

16. A method in a cardiac state system comprising a processing unit and a storage unit where one or many search tools are stored, wherein the method comprises:
receiving, by the processing unit, input signals including parameters from, or related to, one or many registration points or areas within or outside a heart,
processing the input signals in said processing unit, by applying said search tools, to identify points of interest (POI), wherein said POIs are classified according to a rule based model of how the interaction between different tissue and/or hydro mechanical forces in the heart and circulatory system changes during the mechanical chain of events in one heart cycle, to evaluate hydro-mechanical and/or hydro-dynamic functions of the heart, wherein the rule based model is an event and timing function rule based model

17. The method according to claim 16, wherein the rule based model is based on the dynamic adaptive piston pump (DAPP) technology.

18. The method according to claim 16, wherein the method comprises using search tool/tools configured to identify simple landmarks (SLM) in input signals from said POIs, representing easily identifiable heart events.

19. The method according to claim 16, wherein said search tool/tools is further configured to identify at least one heart cycle and one or more main phases of six main phases (MP1-MP6) timely dividing said heart cycle to establish a cardiac state diagram (CSD).

20. The method according to claim 16, wherein said search tool/tools is further configured to search for and identify global and/or regional event markers, patterns and/or group patterns among said POIs to evaluate hydro-mechanical and/or hydro-dynamic functions of the heart.

21. The method according to claim 16, wherein said search tool/tools is further configured to search for event markers, patterns and/or group patterns associated to the motions of the AV-piston and/or the ventricular septum (IVS).

22. The method according to claim 16, wherein the method comprises, by using said rule based model, searching for and analysing local/regional/segmental differences, from two or more registration points associated to the AV-piston motions, before and/or after tension forces within the heart-muscles have evened out any imbalances in the AV-piston's motion pattern.

23. The method according to claim 16, wherein method comprises, by using said rule based model, searching for and analysing global hydromechanical/dynamical functions by input signals from one or more registration points outside the heart.

24. The method according to claim 16, wherein the method comprises, by using said rule based model, searching for and analysing global hydromechanical/dynamical functions of the heart by using two or more input signals, from registration points outside the heart, that more or less reflect counteracting forces that can be used to validate the timing and pattern of events.

25. The method according to claim 16, wherein the method comprises, by using said rule based model, searching for and analysing global/local hydromechanical/dynamical functions of the heart by using two or more input signals, from external and internal registration points including registration points associated to IVS motions.

26. The method according to claim 16, wherein if not all six main phases of a cardiac state diagram (CSD) have been identified, the method comprises iteratively connecting to a reference database (RDB) to identify missing main phase or phases, wherein said reference database (RDB) includes classified data representing complete cardiac state diagrams (CSDs) with global and local event markers, patterns, group patterns and other heart related data and information.

27. The method according to claim 16, wherein if all six main phases have been identified, the method comprises, by using said rule based model, transferring global and local event markers, patterns and group patterns classified with or without score index according to a predetermined classification scheme and other heart related data and information to said reference database (RDB).

28. The method according to claim 16, wherein said cardiac state system comprises a simulator system configured to compare a newly established CSD with previously classified CSDs and other heart related information stored in reference databases (RDB), in order to modulate and simulate what impact different kinds of chemical, electrical or hydromechanical/dynamical parameters and other heart related information have, to provide decision support when e.g. evaluating treatment options.
